(19) United States
(12) Patent Application Publication
(10) Pub. No.: US 2013/0023591 A1
(43) Pub. Date: Jan. 24, 2013

(54) Phenyl-Containing Organopolysiloxane Composition, Raw Cosmetic Material, and Glossy Cosmetic Material

(71) Applicants: Hiroaki Shoji, Tokyo (JP); Yasuhiro Kaneta, Ichihara-shi (JP); Yasue Kanzaki, Chiba-shi (JP); Hidetoshi Kondo, Chiba-shi (JP)

(72) Inventors: Hiroaki Shoji, Tokyo (JP); Yasuhiro Kaneta, Ichihara-shi (JP); Yasue Kanzaki, Chiba-shi (JP); Hidetoshi Kondo, Chiba-shi (JP)

(21) Appl. No.: 13/519,253

(22) PCT Filed: Dec. 27, 2010
(86) PCT No.: PCT/JP2010/073894

§ 371 Date: Oct. 1, 2012

(30) Foreign Application Priority Data
Dec. 28, 2009 (JP) 2009-297442

(51) Int. Cl. A61K 8/891; C07F 7/02

(52) U.S. Cl. 514/772; 556/450

(57) ABSTRACT

To provide a phenyl-containing organopolysiloxane composition that has a high index of refraction of the entire composition and compounding stability in cosmetic formulation, excellent handling performance, and possibility of adjusting viscosity. Another objective is to provide a cosmetic raw material comprising the composition, and a cosmetic material. The invention is a phenyl-containing organopolysiloxane composition that has an index of refraction of the entire composition that is equal to or greater than 1.50 and that has a viscosity of the entire composition at 25° C. in the range of 200 to 500,000 mPa·s.

### TECHNICAL FIELD

The present invention relates to a phenyl-containing organopolysiloxane composition, which is characterized by a high index of refraction, and which, when mixed with a cosmetic material, imparts to this material excellent glitter, luster, glossiness, and which is characterized by having good handling performance and compounding stability, as a cosmetic raw material, due to preservation of the initial high index of refraction and adjustability of viscosity. The invention also relates to a cosmetic raw material comprising the aforementioned phenyl-containing organopolysiloxane composition, and to a cosmetic material that, when combined with the composition, acquires excellent glossiness and pleasant feel to touch. Furthermore, the invention relates to a simple method of manufacturing the aforementioned composition and to a method of adjusting viscosity of the composition.

### BACKGROUND ART

Phenyl-containing silicone resins, represented by silsesquioxane resins and methylphenylpolysiloxane, find wide application as components of cosmetic materials, and it is known that these resins impart gloss to hair, etc. (see, e.g., Patent Reference 1). For example, the methylphenylsiloxane, which in the International Nomenclature of Cosmetic Ingredients (INSI) is defined as a phenyl trimethicone, imparts to the cosmetic material gloss and luster and therefore is widely used in the field of cosmetics (see Patent Reference 2). Furthermore, it is well known that phenyl-containing silicone compounds have a high index of refraction, and that in controlling the value of the index of refraction such high values can be provided by using phenyl groups as substituents in side molecular chains of the polysiloxane (see, e.g., Non-Patent References 1 and 2). In addition, it was suggested in Patent Reference 3 that for providing good transparency and luster, an alkylphenylpolysiloxane, in which the content of phenyl groups constitutes 50% or more, should be an indispensable component of the composition.

However, although phenyl-containing silicone resins possess good transparency and a high index of refraction, it is not so easy to adjust their viscosity, and when this is done by diluting the resins with silicone oil, it is difficult to maintain the index of refraction of the entire composition at a level equal to or higher than 1.50 (in the best case, this index should be in the range of 1.55 to 1.60). Since the index of refraction decreases, it is impossible to impart to the cosmetic material sufficient glossiness. On the other hand, methylphenylpolysiloxane possesses excellent index of refraction. Normally, however, it is difficult to produce methylphenylpolysiloxane with a substitution rate equal to or higher than 50% and with viscosity higher than 200 mPa·s. Furthermore, since the chain-like methylphenylpolysiloxane does not have a three-dimensional cross-linked structure, it may become a liquid having low viscosity and elute from the cosmetic material. When the methylphenylpolysiloxane is added alone, it cannot provide continuous glossiness and satisfactory tactile sensation.

On the other hand, Patent Reference 4 discloses a composition for cosmetic application, in which, in order to acquire glossiness, the composition comprises a propylphenylsilsequioxane resin having weight-average molecular weight in the range of 2,000 to 30,000, a phenylsilicone, or a similar aromatic solvent, and, arbitrarily, a cosolvent. Furthermore, Patent Reference 5 discloses a cosmetic material compounded from a phenylsilicone oil and a phenyl-containing silicone powder.

Since the compositions that contain the aforementioned propylphenylsilsequioxane resin have a high index of refraction, they demonstrate a certain effect in imparting continuous glossiness to cosmetic materials. However, these compositions still need improvement in appearance and tactile sensations. Further, the propylphenylsilsequioxane resins have poor miscibility, in particular with phenyl silicone, and therefore their oil components can easily separate and impair composition uniformity. This, in turn, causes problems associated with storage instability of cosmetic materials compounded with the aforementioned components. Difficulties are also observed in controlling viscosity of the compositions. The systems that use a silicone powder in combination with a phenylsilicone oil have an improved sense of use, but there is still room for improvement from the viewpoint of sense of transparency, index of refraction and appearance characteristics such as glitter and luster. The storage stability also remains inadequate.

[Patent Reference No. 1] Japanese Unexamined Patent Application Publication (hereinafter referred to as “Kokai”) S62-234012

### [Patent Reference No. 2] Kokai H01-168607

### [Patent Reference No. 3] Kokai H07-089844 (JP Patent No. 3207030)

### [Patent Reference No. 4] Kokai 2009-19033

### [Patent Reference No. 5] Kokai 2007-39373

[Non-Patent Reference No. 1] Dow Corning Toray—Catalog: “Silicone for Personal Care”, No. Y517”, P. 13, Issue of Feb. 1, 2009

### [Non-Patent Reference No. 2] Silicone Handbook (Nikkan Kogyo Shimbun Co), Kunio ITO, p. 404)

### SUMMARY OF INVENTION

### Technical Problems to be Solved

The above-described problems are solved by the present invention. It is an object of the present invention to provide a phenyl-containing organopolysiloxane composition that has a high index of refraction of the entire composition, and compounding stability in cosmetic formulation, excellent handling performance, and possibility of adjusting viscosity due to uniform miscibility of a phenylsilsequioxane resin with a phenyl-containing organopolysiloxane. It is another object of the invention to provide a cosmetic raw material that comprises the aforementioned phenyl-containing organopolysiloxane composition and that, when mixed with a cosmetic material, improves tactile characteristics of the latter and imparts to the cosmetic material properties of continuous glossiness, glitter, and luster. It is a further object to provide a cosmetic material, in particular makeup cosmetic material, that comprises the aforementioned phenyl-containing organopolysiloxane composition.

An another object of the invention is to provide a method of producing of the phenyl-containing organopolysiloxane composition, in which various components are provided in an easily miscible state. A further object of the invention is to provide a simple method for adjusting viscosity of the phenyl-containing organopolysiloxane composition, wherein the composition keeps its inherently high index of refraction.

### Solution to Problems

The above objects are achieved by providing a phenyl-containing organopolysiloxane composition that has an index of refraction of the entire composition that is equal to or greater than 1.50 and that has a viscosity of the entire composition at 25° C. in the range of 200 to 500,000 mPa·s.

More specifically, the above objects are achieved by providing a phenyl-containing organopolysiloxane composition comprising: a phenyl silsesquioxane resin (A) that has a number-average molecular weight measured by gel permeation chromatography (GPC) in the range of 500 to 2,000, wherein 15 or more mole % of units in the resin structure comprise phenylsiloxy units represented by the following formula: (C₆H₅SiO_{3/2}); and

a specific amount of a phenyl-containing organopolysiloxane (B) that is liquid at 25° C., has an index of refraction equal to or greater than 1.45. The above objects are also achieved by using the aforementioned phenyl-containing organopolysiloxane composition as a cosmetic raw material and by providing a cosmetic material that comprises the aforementioned phenyl-containing organopolysiloxane composition.

The above-mentioned objects are also achieved by providing a method of producing the phenyl-containing organopolysiloxane composition, the method comprising the steps of: (I) mixing components (A) and (B) in the presence of an organic solvent; and (II) removing the organic solvent from the mixture upon completion of Step (I). The objective is also achieved by adjusting the viscosity of the composition at 25° C. in the range of 200 to 100,000 mPa·s by adjusting the mixture ratio of component (A) to component (B) in the range of (1:0.5) to (1:10).

In other words, the aforementioned objects are achieved by providing the following:

[1] A phenyl-containing organopolysiloxane composition that has an index of refraction of the entire composition that is equal to or greater than 1.50 and that has a viscosity of the entire composition at 25° C. in the range of 200 to 500,000 mPa·s.
[2] The phenyl-containing organopolysiloxane composition according to Item [1], comprising: 100 parts by mass of a phenyl silsesquioxane resin (A) that has a number-average molecular weight measured by gel permeation chromatography (GPC) in the range of 500 to 2,000, wherein 15 or more mole % of siloxy-units in the resin structure is phenylsiloxy units represented by the following formula: (C₆H₅SiO_{3/2}); and 50 to 1,000 parts by mass of a phenyl-containing organopolysiloxane (B) (except for a organopolysiloxane corresponding to component (A)) that is liquid at 25° C., has an index of refraction equal to or greater than 1.45, and contains in its molecule phenylsiloxy units represented by structural formula (1):

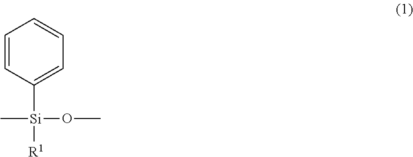

(where, R¹, each independently, is a substituted or unsubstituted univalent hydrocarbon group)
[3] The phenyl-containing organopolysiloxane composition according to Items [2], wherein component (A) comprises a phenyl silsesquioxane resin that has a number-average molecular weight in the range of 750 to 1,800, wherein 40 or more mole % of siloxy-units in the resin structure is phenylsilsesquioxane units represented by the following formula: C₆H₅SiO_{3/2}; and wherein component (B) comprises a phenyl-containing organopolysiloxane represented by the following structural formula (1-1):

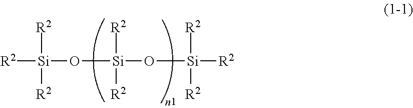

(wherein, R² are selected from phenyl groups, aralkyl groups, hydrogen atoms, hydroxyl groups, or alkyl or fluoro-alkyl groups having 1 to 20 carbon atoms; among these groups, the content of phenyl groups is at least 50% of total groups; and “n1” is a number in the range of 0 to 1000).
[4] The phenyl-containing organopolysiloxane composition according to any Item from [1] to [3], wherein the index of refraction of the entire composition is in the range of 1.50 to 1.60, and wherein the viscosity of the entire composition at 25° C. in the range of 200 to 100,000 mPa·s.
[5] The phenyl-containing organopolysiloxane composition according to any claim from [1] to [4], wherein component (A) comprises a phenyl silsesquioxane resin that has a number-average molecular weight in the range of 750 to 1,800 and that has one or more hydroxyl groups at molecular terminals; and wherein 60 to 100 mole % of siloxy-units in the resin structure is phenylsilsesquioxane units represented by the following formula:

C₆H₅SiO_{3/2}.

[6] The phenyl-containing organopolysiloxane composition according to any Item from [1] to [5], wherein component (B) comprises a phenyldisiloxane, phenyltrisiloxane, or a phenyltetrasiloxane represented by the following structural formula (1-2):

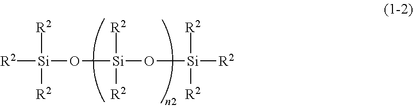

(where R² are the same groups as defined above; among these groups, the content of phenyl group is at least 50%; and “n” is a number in the range of 0 to 2).
[7] The phenyl-containing organopolysiloxane composition according to any Item from [1] to [6], wherein component (B) is a trimethylpentaphenyl trisiloxane.
[8] A cosmetic raw material comprising the phenyl-containing organopolysiloxane composition according to any Item from [1] to [7].
[9] A cosmetic material comprising the phenyl-containing organopolysiloxane composition according to any Item from [1] to [7].
[10] A makeup-type cosmetic material comprising the phenyl-containing organopolysiloxane composition according to any Item from [1] to [7]and at least one type of a powder or a colorant.
[11] A method of producing an phenyl-containing organopolysiloxane composition according to any Item from [1] to [7] comprising the steps of:

(I) mixing components (A) and (B) in the presence of an organic solvent; and

(II) removing the organic solvent from the mixture upon completion of Step (I).

[12] A method of adjusting viscosity of phenyl-containing organopolysiloxane according to any Item from [1] to [7], wherein the viscosity of the composition at 25° C. is adjusted in the range of 200 to 100,000 mPa·s by adjusting the mixture ratio of component (A) to component (B) in the range of (1:0.5) to (1:10).

### Advantageous Effects of Invention

The phenyl-containing organopolysiloxane composition of the invention has an index of refraction of the entire composition that is equal to or greater than 1.50 and that has a viscosity of the entire composition at 25° C. in the range of 200 to 500,000 mPa·s, and since the phenyl silsesquioxane resin and the phenyl-containing organopolysiloxane contained in the composition are uniformly miscible, the composition has an excellent compounding stability in the cosmetic formulation and has a good handling performance. Furthermore, when a cosmetic raw material made from the aforementioned phenyl-containing organopolysiloxane composition is compounded with a cosmetic material, it improves the sense of use of the cosmetic product and imparts to the latter improved glitter, luster, and glossiness. The invention is also efficient in that it provides a cosmetic material that contains the aforementioned phenyl-containing organopolysiloxane, in particular, a glossy makeup-type cosmetic material.

The phenyl-containing organopolysiloxane composition can be easily prepared by the method of the invention since the composition is manufactured when various components of the composition are in an easily and uniformly miscible state.

Furthermore, the invention provides a method for adjusting viscosity of the aforementioned phenyl-containing organopolysiloxane composition that allows maintaining the original high index of refraction (1.50 to 1.60) by appropriately adjusting viscosity of the phenyl-containing organopolysiloxane composition.

### BRIEF DESCRIPTION OF THE DRAWINGS

FIG. 1 is a photograph that illustrates luster and gloss of a trimethylpentaphenyl trisiloxane (on the right) and of the composition of Application Example 1 (on the left) directly after the application.

FIG. 2 is a photograph that illustrates luster and gloss of a trimethylpentaphenyl trisiloxane (on the right) and of the composition of Application Example 1 (on the left) 6 days after the application.

FIG. 3 is a photograph that illustrates luster and gloss of a trimethylpentaphenyl trisiloxane (on the right) and of the composition of Application Example 1 (on the left) 30 days after the application.

### BEST MODE FOR CARRYING OUT THE INVENTION

The composition of the present invention is a phenyl-containing organopolysiloxane composition that has an index of refraction of the entire composition equal to or greater than 1.50 and a viscosity of the entire composition at 25° C. in the range of 200 to 500,000 mPa·s.

More specifically, the composition of the invention is a phenyl-containing organopolysiloxane composition that comprises:

100 parts by mass of a phenyl silsesquioxane resin (A) that has a number-average molecular weight measured by gel permeation chromatography (GPC) in the range of 500 to 2,000, wherein 15 or more mole % of all siloxy units in the resin structure is phenylsiloxy units of the following formula: (C₆H₅SiO_{3/2}); and

50 to 1,000 parts by mass of a phenyl-containing organopolysiloxane (B) (except for a organopolysiloxane corresponding to component (A)) that is liquid at 25° C., has an index of refraction equal to or greater than 1.45, and contains in its molecule phenylsiloxy units represented by structural formula (1) given below. Components (A), (B), a method of preparation of the aforementioned composition, and a method of adjustment of composition viscosity will now be described below in more details.

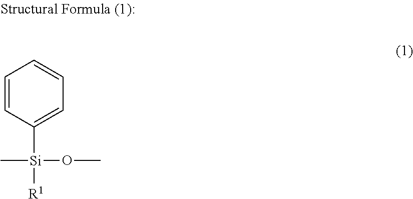

(where, R¹, each independently, is a substituted or unsubstituted univalent hydrocarbon group).

Component (A), which is a specific component of the phenyl-containing organopolysiloxane composition of the present invention, is a phenyl silsesquioxane resin that has a number-average molecular weight measured by gel permeation chromatography (GPC) in the range of 500 to 2,000, wherein 15 or more mole % of siloxy units in the resin structure, which the resin is structured from siloxy units, is phenylsiloxy units of the following formula: C₆H₅SiO_{3/2}. The use of component (A) makes it possible to adjust viscosity of the entire composition to a high value, maintain the initial high index of refraction, and to impart to a cosmetic material improved glossiness and sense of use. Component (A) is a phenyl silsesquioxane resin that has a relatively low molecular weight and is composed mainly of phenylsiloxy (C₆H₅SiO_{3/2}) units. If the number-average molecular weight of the phenyl silsesquioxane resin exceeds the recommended upper unit, then this component will become insufficiently miscible with the phenyl-containing organopolysiloxane of component (B) and, when combined with a cosmetic material, will not provide sufficient effect of dispersion stability and sense of use. On the other hand, if the number-average molecular weight of the aforementioned phenylsilsequioxane resin is lower than the recommended lower limit, the technical effect of improvement in the sense of use and in imparting continuous glossiness to the cosmetic material will be insufficient. In view of the above, it is recommended to have the number-average molecular weight of component (A) in the range of 750 to 1,800, preferably in the range of 850 to 1,750. In particular, the number-average molecular weight in the range of 1,000 to 1,700 is the most optimal for obtaining excellent miscibility with component (B), for imparting continuous glossiness of the cosmetic material, and for realization of high index of refraction.

In component (A), 15 or more mole % of units, from which the resin is structured, comprise phenylsiloxy units of the following formula: (C₆H₅SiO_{3/2}) (herein after referred to as T^{Ph} units). This component is liquid or solid at room temperature and may have a partially branched, network-like, or a cage-like molecular structure. It is recommended that T^{Ph} units constitute more than 40 mole % and preferably 60 to 100 mole % of all siloxy units of component (A). From the viewpoint of a higher index of refraction and technical effect of imparting continuous glossiness to a cosmetic material, it is recommended that the content of T^{Ph} units constitute 65 to 100 mole % of all siloxy units. In the most preferable case, component (A) should comprise a phenylsilsesquioxane resin that consists exclusively of T^{Ph} units. When the index of refraction of the phenyl-containing organopolysiloxane composition of the invention is in the range of 1.55 to 1.60, a content of phenyl groups in component (A) should be equal to or greater than 30 mass % and preferably should be in the range of 40 to 70 mass %. From the viewpoint of improved miscibility with component (B) and other solvents, component (A) should have on its molecular terminals one or more hydroxyl groups (silanol groups). It is preferable that a content of hydroxyl groups in component (B) is in the range of 0.5 to 10 mass %, most preferably in the range of 1.0 to 7.5 mass %.

The phenylsilsesquioxane resin of component (A) may also be combined with siloxy units other than T^{Ph} unit, e.g., with T^{Ph}DQ resin, T^{Ph} resin, T T^{Ph} resin, T^{Ph}Q resin, DT^{Ph} resin, MDT T^{Ph}Q resin, MTT^{Ph}Q resin, MDT^{Ph} resin, M T^{Ph}Q resin composed of arbitrary combinations of tetra-functional siloxy units (Q units), monoorganosiloxy units (T units, except for T^{Ph} units), diorganosiloxy units (D units), and triorganosiloxy units (M units). Substituents groups (organo groups) on silicon atoms of these resins may be represented by hydrogen atoms, alkyl groups with 1 to 8 carbon atoms, alkenyl groups with 2 to 10 carbon atoms, fluoroalkyl groups, or other substituent alkyl groups, long-chain alkyl groups with 9 to 30 carbon atoms, phenyl groups, aryl groups, hydroxyl groups, etc. The use of methyl groups, vinyl group, hydroxyl groups or phenyl groups is preferable from the production point of view. The phenyl silsesquioxane resin of component (A) may be liquid or solid at 25° C., and a flake-like form is preferable for use.

Component (B) is an organopolysiloxane (except for an organopolysiloxane corresponding to component (A) that is liquid at 25° C., has an index of refraction equal to or greater than 1.45, and contains in its molecule phenylsiloxy units represented by structural formula (1) given below. Component (B) is used for adjusting viscosity of the entire composition, for preserving the initially high index of refraction, for imparting luster to a cosmetic material, and for improving transparency and sense of use.

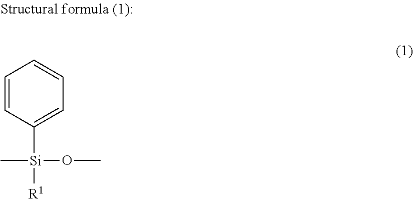

(where, R¹, each independently, is a substituted or unsubstituted univalent hydrocarbon group).

Component (B) is a phenyl-containing organopolysiloxane that has a refraction index equal to or greater than 1.45 and that has predominantly a chain-like or a branched molecular structure. Component (B) may also have a linear molecular structure, provided that this structure satisfies the aforementioned value of the index of refraction and that it is different from the molecular structure of component (A). Although without limitations, the molecular structure may comprise cyclic siloxane such as phenylheptamethyl cyclotetrasiloxane, or 1,1-diphenylheptamethyl cyclotetrasiloxane. If R¹ in the above formula is a group other than a substituted or unsubstituted univalent hydrocarbon group, e.g., a trimethylsiloxy group, this will impair miscibility with component (A) and will not allow obtaining of a uniform phenyl-containing organopolysiloxane. In other words, it is not recommended to use phenyl(trimethylsiloxy)siloxane as component (B).

From the viewpoint of miscibility with component (A) and sense of use of the entire composition, it is preferable to use component (B) in the form of a phenyl-containing organopolysiloxane that is represented by structural formula (1-1) given below. Component (B) that has such a structure may be represented by the following compounds: methylphenylpolysiloxane capped at both molecular terminals with trimethylsiloxy groups, a copolymer of methylphenylsiloxane and dimethylsiloxane capped at both molecular terminals with trimethylsiloxy groups, diphenylpolysiloxane capped at both molecular terminals with trimethylsiloxy groups, a copolymer of diphenylsiloxane and dimethylsiloxane capped at both molecular terminals with trimethylsiloxy groups, and trimethylpentaphenyltrisiloxane.

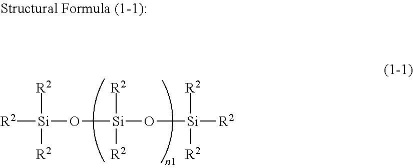

In the above formula, R² designate groups selected from phenyl groups, aralkyl groups, hydrogen atoms, hydroxyl groups, or alkyl groups or fluoroalkyl groups having 1 to 20 carbon atoms. Among these, phenyl groups are used at least in the amount of 50%; “n” is a number in the range of 0 to 1000.

Specific examples of R² groups other than phenyl groups are the following: hydrogen atoms (—H), alkyl groups (e.g., methyl groups, ethyl groups, propyl groups, butyl groups, pentyl groups, hexyl groups, octyl groups, decyl groups, dodecyl groups, octadecyl groups, eicosyl groups, etc.), aralkyl groups (e.g., benzyl groups, phenylethyl groups, etc.), tolyl groups, xylyl groups, cyclohexyl groups, fluoroalkyl groups (trifluoropropyl groups, fluoropropyl groups, or other aforementioned groups, wherein a part or all alkyl groups are substituted with fluorine atoms). Most preferable are methyl groups.

The index of refraction of the phenyl-containing organopolysiloxane used in the present invention should be equal to or greater than 1.435, and preferably should be in the range of 1.50 to 1.60. Normally, the greater is the content of the phenyl groups among all the substituents of the organopolysiloxane of structural formula (1-1), the higher is the index of refraction. Therefore, as mentioned above, it is required that for obtaining the most suitable index of refraction in the range of 1.55 to 1.60, (1-1) the content of phenyl groups in the structure of formula be equal to or greater than 50%. An advantage of using the phenyl-containing organopolysiloxane having an appropriate index of refraction is that it becomes easier to obtain the index of refraction of the entire composition that contains the phenylsilsequioxane of component (A) in the range of high values of 1.50 to 1.60.

Component (B) is liquid at 25° C., and it is preferable that in structural formula (I-1), “n1” is a number in the range of 0 to 1000. From the viewpoint of improved tactile properties and possibility of adjusting viscosity of the entire composition, it is recommended to use two or more types of components (B) with different degrees of polymerization. Normally, component (B) of the present invention has a low degree of polymerization. Specifically, “n1” should be in the range of 0 to 100, preferably in the range of 0 to 25.

Compounds most preferable for use as components (B) of the invention are phenyldisiloxanes, phenyltrisiloxanes, or phenyltetrasiloxanes represented by structural formula (1-2) shown below. In this formula, R² designates the same groups as defined above. Among these groups, the content of phenyl groups is at least 50%; and “n2” is a number in the range of 0 to 2.

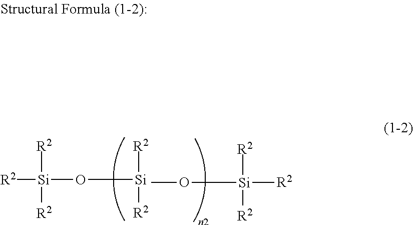

The aforementioned phenyldisiloxanes, phenyltrisiloxanes, or phenyltetrasiloxanes of structural formula (1-2) are characterized by viscosity that is below 250 mPa·s at 25° C., and by an index of refraction that is equal to or higher than 1.55. By utilizing such properties, it becomes possible to provide a cosmetic raw material that is free of stickiness, prevents powdery deposition of an inorganic powder, when the latter is combined with the cosmetic material, improves transparency, and provides natural luster free from glitter of skin or hair.

Examples of the aforementioned phenyldisiloxanes, phenyltrisiloxanes, and phenyltetrasiloxane of structural formula (1-2) are given below. It is understood, however, that these examples should not be construed as limitative. Most suitable for use as component (B) is trimethylpentaphenyltrisiloxane.

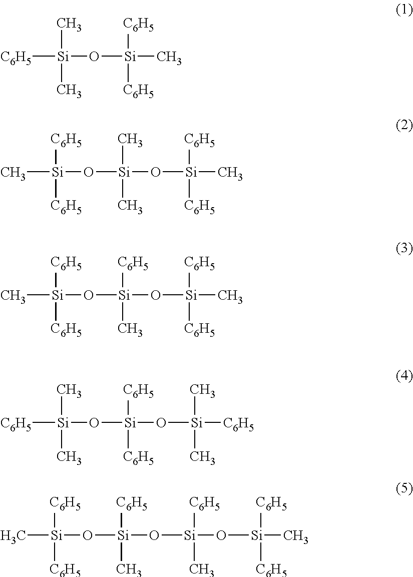

The aforementioned phenyl-containing organopolysiloxanes can be synthesized by methods known in the art. For example, in case of a compound represented by aforementioned formula (2), these organopolysiloxanes can be obtained by carrying out a dehydrochlorination condensation reaction with subsequent distillation and purification. Another method consists of subjecting 1,3-dimethyl-1,1,3,3-tetraphenyldisiloxane and 1,1,3,3,5,5,7,7-octamethylcyclotetrasiloxane to an equilibration reaction in the presence of an acid or an alkali catalyst and then conducting distillation and purification.

It is preferable that the phenyl-containing organopolysiloxane used in the present invention be purified to the level at which the product satisfies all the properties mentioned above. Therefore, selection of a production method resulting in a broad distribution of molecular weight is favorable for purification and distillation. However, selection of a production method resulting in a narrow molecular-weight distribution and, in this case without purification, is also acceptable for use.

The composition of the invention is a phenyl-containing organopolysiloxane composition that has an index of refraction of the entire composition that is equal to or greater than 1.50 and that has a viscosity of the entire composition at 25° C. in the range of 200 to 500,000 mPa·s. The composition is characterized by comprising 100 parts by mass of the aforementioned phenyl silsesquioxane resin (A) and 50 to 1,000 parts by mass of a phenyl-containing organopolysiloxane (B) (except for a organopolysiloxane corresponding to component (A)) that has an index of refraction equal to or greater than 1.45. As can be seen from the above, both components (A) and (B) contain phenyl groups and have high indices of refraction. Therefore, it becomes possible to easily obtain a high index of refraction of the entire composition, which, specifically, is equal to or greater than 1.50 and preferably is in the range of 1.50 to 1.60. In particular, when a cosmetic raw material having a high index of refraction is used, it is recommended to have the index of refraction of the entire composition in the range of 1.55 to 1.60, preferably in the range of 1.56 to 1.59. It is also preferable from the viewpoint of improved compounding stability and handling performance of the raw cosmetic material to adjust the viscosity of the entire composition to the range of 200 to 100,000 mPa·s.

Selection of the component (A) in the form of a phenyl silsesquioxane resin that has a relatively low weight-average molecular weight, contributes to improvement in miscibility with component (B) in preparation of the composition and significantly improves sense of use when the composition is used as a cosmetic raw material. Furthermore, by selecting component (B) in the form of a phenyl-containing organopolysiloxane that has low viscosity and high index of refraction, it becomes possible to provide the index of refraction in the range of 1.56 to 1.59 and to obtain viscosity of the entire composition in the range of 200 to 100,000 mPa·s at 25° C.

More specifically, the phenyl-containing organopolysiloxane having an index of refraction of the entire composition in the range of 1.55 to 1.60 and viscosity of the entire composition in the range of 200 to 100,000 mPa·s at 25° C. can be easily obtained by providing the compounding ratio of components (A) and (B) in the range of (1:0.5) to (1:10). As it has been exemplified above, when a disiloxane or trisiloxane having viscosity equal to or lower than 250 mPa·s at 25° C. is used as component (B), it becomes possible, by adjusting the compounding ratio of components (A) and (B) to the range of (1:0.5) to (1:10), to easily obtain an arbitrary composition with a high index of refraction and with viscosity in a wide range between low and high.

For example, a composition having viscosity of the entire composition in the range of 500 to 1000 mPa·s at 25° C. can be obtained when in the phenylsilsequioxane resin utilizing component (A) in the form of the phenylsilsesquioxane with a number-average molecular weight of 1500 and component (B) in the form of the trimethylpentaphenyltrisiloxane (index of refraction: 1.58; viscosity: 175 mPa·s), component (B) is used in an amount of 400 parts by mass per 100 parts by mass of component (A). When component (B) is added in the amount of 150 parts by mass, the composition will be obtained with viscosity of the entire composition in the range of 25,000 to 26,000 mPa·s at 25° C. On the other hand, if component (B) is added in the amount of 65 parts by mass, the composition will be obtained with viscosity of the entire composition equal to or greater than 30,000 mPa·s at 25° C. Thus, such compositions will have different entire-composition viscosities, but, their values of index of refraction will be in the range of 1.57 to 1.58 in general.

There are no special restrictions with regard to the means that can be used for obtaining the phenyl-containing organopolysiloxane composition of the invention by uniformly mixing the aforementioned components, and the components can be uniformly mixed and kneaded at room temperature with the use of such means as a ball mill, a vibrating mill, a kneader-mixer, a screw-type extruder, a paddle mixer, a ribbon mixer, a Banbury mixer, a Ross mixer, a Henschel mixer, a flow-jet mixer, a Hobart mixer, a roll mixer, or any other conventional mixing or kneading device. In order to improve efficiency and uniformity of mixing the phenylsilsesquioxane of component (A) and the phenyl-containing organopolysiloxane of component (B), the components can be mixed at a temperature equal to or greater than 50° C. It is preferable to mix the components at a temperature in the range of 50 to 150° C., and most preferably, at a temperature equal to or greater than 70° C.

In order to obtain uniformly-mixed phenyl-containing organopolysiloxane composition, components (A) and (B) should be mixed in the presence of an organic solvent. However, since an organic solvent reduces the index of refraction, it should be removed from the mixture system by known means, e.g., by stripping. Specifically, it is preferable that the content of an organic solvent in the obtained phenyl-containing organopolysiloxane composition do not exceed 3 mass %, more preferably be in the range of 0 to 2 wt. %, and most preferably be practically close to 0%.

The aforementioned organic solvent can be exemplified by methanol, ethanol, isopropyl alcohol, or a similar low-alkyl alcohol having 1 to 6 carbon atoms; hexane, heptane, octane, nonane, or a similar aliphatic hydrocarbon; and benzene, tolyene, xylene, or a similar aromatic hydrocarbon. Stripping is facilitated if the organic solvent used in the manufacture of the aforementioned composition is added in an amount in the range of 10 to 1000 parts by mass, preferably 10 to 200 parts by mass per 100 parts by mass of the sum of components (A) and (B).

Although the phenyl-containing organopolysiloxane composition of the invention consists essentially of components (A) and (B), the composition may also contain a powder (C) or a coloring agent. Mixing of components (A) and (B) with such a powder or a coloring agent, in particular with a powder, and specifically with in inorganic powder or a pearl pigment, may form a cosmetic raw material that produces an improved sense of transparency, and imparts to skin and hair a natural luster and brightness with no glare.

Component (C) is a powder or a colorant, mainly one used in conjunction with cosmetics. If such a powder or colorant is used, its particles may have any shape, such as a spherical shape, a rod-like shape, a needle-like shape, a plate-like shape, an irregular shape, a spindle-like shape, etc. Similarly, the particles may have diameters corresponding to fumed type particles, microparticles, pigment-type particles, etc.; they may have different structures (porous, non-porous structure, etc.). When the powder or colorant is incorporated into a cosmetic as a pigment, the powder may be represented by one or more types of pigments having the average diameter in the range of 1 nm to 20 μm and selected from an inorganic pigment powder, organic pigment powder, or a resin powder.

The powders or coloring agents can be exemplified by inorganic powders, organic powders, surface-active metal salt powders (metal soaps), colored pigments, pearl pigments, silicone-type powders, silicate clay minerals modified with organic materials, metal powder pigments, etc. These pigments can be used in combinations.

Specific examples of inorganic powders are the following: titanium oxide, zirconium oxide, zinc oxide, cerium oxide, magnesium oxide, barium sulfate, calcium sulfate, magnesium sulfate, calcium carbonate, magnesium carbonate, talc, mica, kaolin, sericite, white mica, synthetic mica, phlogopite, lepidolite, biotite, lithia mica, silicic acid, silicic anhydride, aluminum silicate, sodium silicate, sodium magnesium silicate, magnesium silicate, aluminum magnesium silicate, calcium silicate, barium silicate, strontium silicate, metal salts of tungstenic acid, hydroxyapatite, vermiculite, higilite, bentonite, montmorillonite, hectolitre, zeolite, ceramics powder, calcium secondary phosphate, alumina, aluminum hydroxide, boron nitride, etc.

Organic powders include polyamide powder, polyester powder, polyethylene powder, polypropylene powder, polystyrene powder, polyurethane, benzoguanamine powder, polymethyl benzoguanamine powder, polyterafluoroethylene powder, polymethyl methacrylate powder, cellulose, silk powder, nylon powder, Nylon 12, Nylon 6, styrene/acrylic acid copolymer, divinylbenzene/styrene copolymer, vinyl resin, urea resin, phenol resin, fluororesin, silicone, acrylic resin, melamine resin, epoxy resin, polycarbonate resin, microcrystalline fiber powder, starch powder, and lauroyl lysine, etc.; the surface activating metal salt powders (metal soaps) include zinc stearate, aluminum stearate, calcium stearate, magnesium stearate, zinc myristate, magnesium myristate, zinc palmitate, zinc laurate, zinc cetyl phosphate, calcium cetyl phosphate, and zinc/sodium cetyl phosphate; examples of the colored pigments include inorganic red pigments such as iron oxide, iron hydroxide, and iron titanate, inorganic brown pigments such as γ-iron oxide, inorganic yellow pigments such as iron oxide yellow and loess, inorganic black pigments such as iron oxide black and carbon black, inorganic violet pigments such as manganese violet and cobalt violet, inorganic green pigments such as chromium hydroxide, chromium oxide, cobalt oxide, and cobalt titanate; inorganic blue pigments such as Prussian blue and ultramarine blue, lakes of tar pigments, lakes of natural dyes, and synthetic resin powder complexes thereof; examples of the tar pigments include Red No. 3, Red No. 104, Red No. 106, Red No. 201, Red No. 202, Red No. 204, Red No. 205, Red No. 220, Red No. 226, Red No. 227, Red No. 228, Red No. 230, Red No. 401, Red No. 505, Yellow No. 4, Yellow No. 5, Yellow No. 202, Yellow No. 203, Yellow No. 204, Yellow No. 401, Blue No. 1, Blue No. 2, Blue No. 201, Blue No. 404, Green No. 3, Green No. 201, Green No. 204, Green No. 205, Orange No. 201, Orange No. 203, Orange No. 204, Orange No. 206, and Orange No. 207; examples of the natural dyes include carminic acid, laccaic acid, carthamin, brazilin, and crocin.

Examples of pearl pigment include titanium oxide-coated mica, titanium mica, ferrous oxide coated mica, bismuth oxychloride, titanium oxide coated bismuth oxychloride, titanium oxide coated talc, fish scales, and titanium oxide-coated colored mica; and examples of a usable metallic powder pigment include aluminum powder, copper powder and stainless powder.

Clay minerals modified with organic materials include dimethylbenzyldodecyl ammonium montmorillonite clay, dimethyldioctadecyl ammonium montmorillonite clay, dimethylalkyl ammonium hectolite, benzyldimethyl stearyl ammonium hectolite, aluminum magnesium silicide coated with distearyldimethyl ammonium chloride, etc. These compounds are commercially available products of National Lead Co., Inc., such as Benton-27 (hectolite treated with benzyldimethylstearyl ammonium chloride; Benton-38 (hectolite treated with distearyldimethyl ammonium chloride,), etc.

Silicone-based powders include silicone powder, spherical silicone rubber powder, and spherical silicone rubber powder surface-coated with polymethylsilsesquioxane. Most suitable is the spherical silicone rubber powder having the primary powder diameter in the range of 0.1 to 50 μm. These powders are available as commercial products of Dow Corning Toray Co., Ltd., such as Torayfil E-506S, Torayfil E-508, 9701 Cosmetic Powder, 9702 Powder, etc. The spherical silicone powder may be in the form of an aqueous dispersion and may be directly used as a cosmetic material. These aqueous dispersions are commercially available from Dow Corning Toray Co., Ltd. as BY 29-129, PF-2001 PIF Emulsion, etc.

It is recommended to subject the aforementioned powders or colorants to a water-repellant treatment. Furthermore, it is possible to combine the powders with colorants and/or to combine different colorants, or to add conventional oil agents, silicones other than the organopolysiloxanes of the invention, as well as fluoro-compounds, to subject the powders or coloring agents to surface treatment with surface-active substances, and, if necessary, to combine the powders and colorants of two or more different types.

Examples of the aforementioned water-repellant treatment of the powders and/or coloring agents are the following: treatment with methylhydrogenpolysiloxane, treatment with silicone resin, treatment with silicone gum, treatment with acrylic silicone, treatment with fluorosilicone, or with other organosiloxanes; treatment with zinc stearate, or other metal soaps, treatment with silane coupling agents, treatment with alkyl silane, and with other similar silanes; treatment with perfluoroalkylsilane, perfluoroalkylphosphate, perfluoropolyester, or with another fluoro compound; treatment with N-lauroyl-L-lysine, or with another amino acid; treatment with squalane, or with another oil agent; treatment with acrylacrylate, or with another acryl compound, etc. Two or more such treatments can be combined.

The phenyl-containing organopolysiloxane composition of the invention is prepared from aforementioned components (A) and (B), but arbitrarily the composition may also contain aforementioned component (C). Furthermore, within the limits that is not in conflict with the objects of the present invention, especially when the composition is used as raw cosmetic material, the composition may also incorporate various components, which will be described later.

Since the phenyl-containing organopolysiloxane composition of the invention provides high index of refraction of the entire composition and uniform miscibility of the phenylsilsesquioxane resin with phenyl-containing organopolysiloxane, the composition can be used as a cosmetic raw material that imparts to cosmetic products excellent compounding stability and handling performance. When compounded with a cosmetic material, the above-described cosmetic raw material imparts to this cosmetic material such properties as durability, water-proof properties, affinity to skin, water-repellant properties, softness, water vapor permeability, gas permeability, film-forming properties, filler holding properties, and lubricity. The composition also imparts to the cosmetic material improved sense of use with no stickiness. In particular, when used for the preparation of cosmetics, the aforementioned cosmetic raw material improves transparency of the cosmetic material, continuous glossiness, brightness, and luster. When compounded with an inorganic powder or pearl pigment, the composition suppresses floating and imparts to skin and hair natural gloss free from glittering.

The phenyl-containing organopolysiloxane composition improves sense of transparency of the cosmetic material that contains this composition, and, when it is applied onto skin or lips, it produces the effect of luster and gloss. Therefore, the composition is especially suitable for use with a makeup cosmetic where brightness, glossy appearance and sense of transparency are required.

An amount in which the phenyl-containing organopolysiloxane composition is to be added to a cosmetic material has to be in harmony with the type, conditions, desired properties, and other components of the cosmetic material, and is selected in accordance with the prescription, but in case of a makeup cosmetics, the content of the phenyl-containing organopolysiloxane composition should be in the range of 0.5 to 50 mass %, preferably 1.0 to 20 mass %.

In addition to the aforementioned phenyl-containing organopolysiloxane composition, the cosmetic material of the invention may incorporate one or more components selected from the same powders and coloring agents as aforementioned component (C), oil agents (D) (in particular, waxes and volatile oil agents), oil thickeners and gelling agents (E), surface-active agents (F), film-forming agents (G), water-soluble polymers (H), UV blockers (I), lower univalent alcohols (J), polyhydric alcohols (K), and water (L). Blending with these components is especially suitable for preparation of makeup cosmetics. The amounts in which the above components are added can be selected in accordance with conventional formulations or prescriptions of cosmetic products.

Powders and colorants used as component (C) are the same as defined above. Component (C) can be added to the cosmetic material as a component of the phenyl-containing organopolysiloxane composition, or two or more types of the powders and colorants can be added to the cosmetic material independently. It is recommended that the powder or colorant be added in an amount of 5 to 50 mass % but this range should not be construed as limiting and other quantities can be added depending on the type and prescription of the cosmetic material. The use of pearl pigment and coloring pigment, or combination of both is especially advantageous when the cosmetic material of the invention is a makeup cosmetic for lips, area around the eye, or eyelashes.

The component (D) may be exemplified by an oil agent, such as a waxy oil agent or an oily-form agent, that does not correspond to the aforementioned phenyl-containing organopolysiloxane composition. When the cosmetic material of the invention is appropriately used as a makeup cosmetic for lips, area around the eye, or eyelashes, it is recommended to combine this cosmetic with a waxy oil agent, a volatile oil agent, or with an oil agent obtained by combining both aforementioned agents.

The oil agent of component (D) may be selected, e.g., from silicone oil, hydrocarbon oil, ester oil, various vegetable oils, animal oils and fats, higher alcohols, liquid fatty acids, triglyceride, and artificial sebum. These oils can be used individually or in combinations of two or more.

The silicone oil is a component that corresponds neither to component (A), nor to component (B), nor to the mixture of both these components. Specific examples of the silicone oil are the following: cyclic organopolysiloxanes such as hexamethylcyclotrisiloxane (D3), octamethylcyclotrisiloxane (D4), decamethylcyclopentasiloxane (D5), dodecamethylcyclohexasiloxane (D6), 1,1-diethylhexamethylcyclotetrasiloxane, 1,3,5,7-tetravinyltetramethylcyclotetrasiloxane, 1,3,5,7-tetramethylcyclotetrasiloxane, 1,3,5,7-tetracyclohexyltetramethylcyclo-tetrasiloxame, tris(3,3,3-trifluoropropyl)trimethylcyclotrisiloxane, 1,3,5,7-tetra(3-methacryloxypropyl)tetramethylcyclotetrasiloxane, 1,3,5,7-tetra(3-acryloxypropyl)tetramethylcyclotetrasiloxane, 1,3,5,7-tetra(3-carboxypropyl)tetramethylcyclotetrasiloxane, 1,3,5,7-tetra(3-vinyloxypropyl)tetramethylcyclotetrasiloxane, 1,3,5,7-tetra(p-vinylphenyl)tetramethylcyclotetrasiloxane, 1,3,5,7-tetra [3-(p-vinylphenyl)propyl]tetramethylcyclotetrasiloxane, 1,3,5,7-tetra[N-acryloyl-N-methyl-3-aminopropyl]tetramethylcyclotetrasiloxane, 1,3,5,7-tetra(N,N-bis(lauroyl)-3-aminopropyl)tetramethylcyclotetrasiloxane, etc. The linear-chain organopolysiloxane may be exemplified by dimethylpolysiloxane capped at both molecular terminals with dimethylpolysiloxy groups (i.e., a dimethylsiloxane with viscosity from low viscosity of 2 cst or 6 cst to high viscosity of about 1,000,000 cst), organohydrogenpolysiloxane, methylalkylpolysiloxane having both molecular terminals capped with trimethylsiloxy groups, a copolymer of methylalkylsiloxane and dimethylpolysiloxane having both molecular terminals capped with trimethylsiloxy groups, a copolymer of methyl(3,3,3-trifluoropropyl)siloxane and dimethylsiloxane having both molecular terminals capped with trimethylsiloxy groups, α, ω-dihydroxypolydimethylsiloxane, α,ω-diethoxypolydimethylsiloxane, 1,1,1,3,5,5,5-heptamethyl-3-octyltrisiloxane, 1,1,1,3,5,5,5-heptamethyl-3-dodecyltrisiloxane, 1,1,1,3,5,5,5-heptamethyl-3-hexadecyltrisiloxane,tris-trimethylsiloxymethylsilane, tris-trimethylsiloxyalkylsilane, tetrakis-trimethylsiloxysilane, tetramethyl-1,3-dihydroxydisiloxane, octamethyl-1,7-dihydroxytetrasiloxane, hexamethyl-1,5-diethoxytrisiloxane, hexamethyldisiloxane, octamethyltrisiloxane, alkyl-modified silicone, higher alcohol-modified silicone, higher fatty acid-modified silicone, etc.

The hydrocarbon oil of component (D) includes liquid paraffin, light liquid isoparaffin, heavy liquid isoparaffin, Vaseline, n-paraffin, isoparaffin, isododecane, isohexadecane, polyisobutylene, hydrogenated polyisobutylene, polybutene, hydrogenated polybutene, ozokerite, ceresin, microcrystalline wax, paraffin wax, polyethylene wax, polyethylene-polypropylene wax, squarane, squarene, pristane, polyisoprene, etc.

Examples of the ester oil include hexyldecyl octanoate, cetyl octanoate, isopropyl myristate, isopropyl palmitate, butyl stearate, hexyl laurate, myristyl myristate, oleyl oleate, decyl oleate, octyldodecyl myristate, hexydecyl dimethyloctanoate, cetyl lactate, myristyl lactate, diethyl phthalate, dibutyl phthalate, lanolin acetate, ethyleneglycol monostearate, propyleneglycol monostearate, propyleneglycol dioleate, glyceryl monostearate, glyceryl monooleate, glyceryl-tri-2-ethylene hexane, trimethylolpropane tri-2-ethylhexane, ditrimethylolpropane triethylhexanoate, ditrimethylolpropane isostearate/sebacate, trimethylolpropane trioctanoate, trimethylolpropane triisosterate, diisopropyl adipate, diisobutyl adipate, 2-hexyldecyl adipate, di-2-heptylundecyl adipate, diisostearyl malate, hydrogenated castor oil monoisostearate, N-alkylglycol monoisostearate, octyldodecyl isostearate, isopropyl isostearate, isocetyl isostearate, ethyleneglycol di-2-ethylenehexanoate, heptaerythrritol tetra-2-ethylhexanoate, octyldodecyl γ-ester, ethyl oleate, octyldodecyl oleate, neopentylglycol dicaproate, triethyl citrate, 2-ethylhexyl succinate, dioctyl succinate, isocetyl stearate, isopropyl sebacate, diisopropyl sebacate, di-2-ethylhexyl sebacate, diethyl sebacate, dioctyl sebacate, dibutyloctyl sebacate, cetyl palmitate, octyldecyl palmitate, octyl palmitate, 2-ethylhexyl palmitate, 2-hexyldecyl palmitate, 2-heptylundecyl palmitate, cholesteryl 12-hydroxystearate, dipentaerythritol fatty acid ester, 2-hexyldecyl myristate, ethyl laurate, 2-octyldodecyl N-lauroyl-L-glutamate, di(cholesteryl/behenyl/octyldodecyl)N-lauroyl-L-glutamate, di(cholesteryl/octyldodecyl)N-lauroyl-L-glutamate, di(phytosteryl/behenyl/octyldodecyl)N-lauroyl-L-glutamate, di(phytosteryl/octyldodecyl)N-lauroyl-L-glutamate, N-lauroylsarcosine isopropyl, diisostearyl malate, neopentylglycol dioctanate, isodecyl neopentanoate, isotridecyl neopentanoate, isostearyl neopentanoate, isononyl isononanoate, isotridecyl isononanoate, octyl isononanoate, isotridecyl isononanoate, diethylpentane diol dineopentanoate, methylpentane diol dineopentanoate, octyldodecyl neodecanoate, octyldodecyl neodecanoate, 2-butyl-2-ethyl-1,3-propane diol dioctanoate, pentaerythritol tetraoctanoate, hydrogenated rosinepentaerythritol, pentaerythrityl tetraethylhexanoate, dipentaerythrityl hydroxystearate/stearate/rosinate, polyglyceryl tetraisostearate, polyglyceryl-10 nanoisostearate, polyglyceryl-8 deca(erucate/isostearate/ricinoleate), diglyceryl oligoester (hexyldecanoate/sebacate), glycol (ethyleneglycol distearate) distearate, diisopropyl dimer dilinoleate, diisostearyl dimer dilinoleate, di(isostearyl/behenyl)dimer dilinoleate, (phytosteryl/behenyl)dilinoleate, (phitosteryl/isostearyl/cetyl/stearyl/behenyl)dilinoleate, dimer dilinoleyl dimmer dilinoleate, dimer dilinoleyl diisostearate, dimer dilinoleyl hydrogenated rosin condensate, hydrogenated castor oil dimer dilinoleate, hydroxyalkyl dimmer dilinoleyl ester, glyceryl triisooctanoate, glyceryl triisostearate, glyceryl trimyristate, glyceryl triisopalmitate, glyceryl trioctanoate, glyceryl trioleate, glyceryl diisostearate, (caprylic/caprylic) triglyceride, (caprylic/caprylic/myristic/stearic) triglyceride, (hydrogenated ester gum) hydrogenated rosin triglyceride, rosin triglyceride (ester gum), (behenic acid/eicosanoic diacid) glyceryl, di-2-heptyl undecanoic acid glyceryl, myristic/isostearic diglyceryl, cholestryl acetate, cholesteryl nanoate, cholesteryl stearate, cholesteryl isostearate, cholesteryl oleate, cholesteryl 12-hydroxystearate, macademia nut fatty acid cholesteryl, macademia nut fatty acid phytostearyl, phytostearyl isostearate, soft lanolin fatty acid cholesteryl, hydrogenated lanolin fatty acid cholesteryl, long chain/branched fatty acid cholesteryl, long-chain α-hydroxy fatty acid cholesteryl, octyldodecyl linoleate, octyldodecyl lanolin fatty acid, octyldodecyl erucate, hydrogenated castor oil isostearate, avocado oil fatty acid ethyl, lanolin fatty acid isopropyl, etc.

The natural vegetable oils, animal oils and fats and semi-synthetic oils and fats include avocado oil, flax seed oil, almond oil, Ibota wax, perilla oil, olive oil, cacao butter, kapok wax, kaya oil, carnauba wax, cod liver oil, candelilla wax, beef tallow, neat's-foot oil, beef bone fat, hydrogenated beef tallow, apricot kernel oil, spermaceti wax, hydrogenated oil, wheat germ oil, sesame oil, rice germ oil, sugar cane wax, sasanqua oil, safflower oil, shear butter, Chinese tung oil, cinnamon oil, jojoba wax, olive squalane, shellac wax, turtle oil, soybean oil, tea seed oil, camellia oil, evening primrose oil, corn oil, lard, rapeseed oil, Japanese wood oil, rice bran oil, germ oil, horse fat, persic oil, palm oil, palm kernel oil, castor oil, hydrogenated castor oil, castor oil fatty acid methyl ester, sunflower oil, grape oil, bayberry wax, jojoba oil, macadamia nut oil, beeswax, mink oil, cottonseed oil, cotton wax, Japanese wax kernel oil, montan wax, coconut oil, hydrogenated coconut oil, tri-coconut oil fatty acid glyceride, mutton tallow, peanut oil, lanolin, liquid lanolin, hydrogenated lanolin, lanolin alcohol, hard lanolin, lanolin acetate, isopropyl lanolate, POE lanolin alcohol ether, POE lanolin alcohol acetate, lanolin fatty acid polyethylene glycol, POE hydrogenated lanolin alcohol ether, egg yolk oil, etc.

The higher alcohols include, e.g., the following: lauryl alcohol, myristyl alcohol, palmityl alcohol, stearyl alcohol, behenyl alcohol, hexadecyl alcohol, oleyl alcohol, isostearyl alcohol, hexyldodecanol, octyl dodecanol, cetostearyl alcohol, 2-decyltetradecinol, cholesterol, sitosterol, phytosterol, lanosterol, POE cholesterol ether, monostearyl glycerin ether (batyl alcohol), and monooleyl glyceryl ether (cerakyl alcohol), isostearyl glyceryl ether, etc.

The higher fatty acids include, e.g., lauric acid, myristic acid, palmitic acid, stearic acid, behenic acid, undecylenic acid, oleic acid, linoleic acid, linolenic acid, arachidonic acid, eicosapentaenoic acid (EPA), docosahexaenoic acid (DHA), isostearic acid, 12-hydroxystearic acid, etc.

Among the above oils, silicone oil, such as decamethyl cyclopentasiloxane (D5), dodecamethylcyclohexasiloxane (D6), or a similar volatile silicone oil, should be added to cosmetic material when it is necessary to impart to the cosmetic material a refreshing sense of use. Similarly, addition to a cosmetic material of volatile hydrocarbon oils, such as light isoparaffin, isododecane, or similar volatile hydrocarbon oils, also is recommended for impart a refreshing sense of use.

Furthermore, the following oils can be selected and added individually or in combinations of two or more to makeup cosmetics, especially to makeup cosmetic for lips, area around the eyes, and eyelashes, when such a makeup is used as a basis for cosmetic materials of the invention: polybutene, hydrogenated polybutene, paraffin wax, Vaseline, lanolin, beewax, carnauba wax, candelilla wax, stearyl alcohol, lauric acid, myristic acid, palmitic acid, stearic acid, isostearic acid, behenic acid, lanolin fatty acid, hydrogenated castor oil, or the like.

The aforementioned oil agents should be used in an amount of 10 to 80 mass % of the total cosmetic material composition. This range, however, should not be construed as limitative and the actual content may depend on the type of the cosmetic material and cosmetic prescription.

Component (E) is a thickener/gelling component for the oil agent and is intended for thickening the composition or for imparting to it a stable form intermediate between liquid and solid and viscoelastic properties. It is recommended to use this component according to the prescription and to use it for arbitrarily controlling the state of the composition and for converting it from liquid into cream, paste, gel, or a solid substance. The thickener/gelling component is added in an amount that depends on the desired state of the composition, but in general the added amount should be in the range of 1 to 20 mass %. Normally, if the thickener/gelling agent is added in an amount smaller than the recommended lower limit, the thickening/gelling effect will be insufficient. The thickener/gelling agent is added in an amount exceeding the recommended upper limit, this will impart to the cosmetic material a sense of heaviness and will impair the sense of use.

Component (E) can be exemplified, by a gelling agent selected, e.g., from the following compounds: derivatives of α,γ-di-n-butylamine, lauroyl-L-glutaminic acid, etc.; dextrin palmitic acid ester, dextrin stearic acid ester, dextrin 2-ethylhexanoic acid palmitic acid ester, or a similar dextrin fatty acid ester; sucrose pulmitic acid ester, sucrose stearic acid ester, or a similar sucrose fatty acid ester; inulin stearic acid ester, fructooligosaccharide 2-ethylhaxanoic acid ester, or a similar fructooligosucrose fatty acid ester; mono-benzylidene sorbitol, or a similar benzylidene derivative of sorbitol.

Component (F) is a surface-active agent. This component may comprise one type or several combined types of surface-active agents selected from anionic, cationic, non-ionic (including silicone type), amphoteric, or semi-polar surface-active agents. In cosmetics, these surface agents are suitable for use as cleaning agent components, antibacterial components, as well as dispersant and emulsifiers for oils.

More specifically, anionic surface-active agents may include saturate or unsaturated fatty acid salts (such as sodium laurate, sodium stearate, sodium oleate, sodium linolate, etc.), alkyl sulfate, alkyl benzene sulfonic acid (such as, e.g., hexyl benzene sulfonic acid, toctyl benzene sulfonic acid, dodecyl hexane sulfonic acid, etc.), or salts thereof, polyoxyalkylene alkyl ether sulfate, polyoxyalkylene alkenyl ether sulfate, polyoxyethylene alkyl ether sulfate, polyoxyethylene alkyl sulfate ester salt, sulfosuccinic acid alkyl ester salt, polyoxyalkylene sulfosuccinic acid alkyl ester salt, polyoxyalkylene alkyl phenyl ether sulfonate, alkane sulfonate, octyltrimethyl ammonium hydroxide, dodecyltrimethyl ammonium hydroxide, alkyl sulfonate, polyoxyethylene alkylphenyl ether sulfonate, polyoxyalkylene alkyl ether acetate, alkyl phosphate, polyoxyalkylene alkyl ether phosphate, acyl glytamate, α-acyl sulfonate, alkyl sulfonate, alkylallyl sulfonate, α-olefin sulfonate, alkyl naphthalene sulfonate, alkane sulfonate, alkyl or alkenyl sulfate, alkyl amide sulfate, alkyl or alkenyl phosphate, alkyl amide phosphate, alkyloyl alkyl taurine phosphate, N-acylamino acid salt, sulfosuccinate, alkyl ether carboxylate, amido ether carboxylate, α-sulfoaliphatic ester salt, alanine derivative, glycine derivative, and arginine derivative. The salts may be represented by sodium salts or similar alkali metal salts, magnesium salts or similar alkali earth metal salts, triethanol amine salts or similar alkanolamine salts, as well as ammonium salts.

The cationic surface-active agents include alkyl trimethyl ammonium chloride, stearyl trimethyl ammonium chloride, lauryl trimethyl ammonium chloride, cetyl trimethyl ammonium chloride, beef tallow alkyl trimethyl ammonium chloride, behenyl trimethyl ammonium chloride, stearyl trimethyl ammonium bromide, behenyl trimethyl ammonium bromide, distearyl dimethyl ammonium chloride, dicocoyl dimethyl ammonium chloride, dioctyl dimethyl ammonium chloride, di(POE) oleyl methyl ammonium chloride (2EO), benzalkonium chloride, alkyl benzalkonium chloride, alkyl dimethyl benzalkonium chloride, benzethonium chloride, stearyl dimethyl benzethonium chloride, quaternary ammonium of lanolin derivative, diethyl amino ethyl amide stearate, diethyl amino propyl amide stearate, behenic acid amidopropyl dimethylhydroxypropyl ammonium chloride, stearoyl colaminoformyl methylpyridinium chloride, cetyl pyridinium chloride, tall oil alkyl benzyl hydroxyethyl imidazolinium chloride, and benzyl ammonium salt.

The nonionic surface-active agents include the following: polyoxyalkylene ethers, polyoxyalkylenealkyl ethers, polyoxyalkylene fatty acid esters, polyoxyalkylene fatty acid diesteres, polyoxyalkylene resin acid esters, polyoxyalkylene (hydrogenated) castor oils, polyoxyalkylene alkylphenols, polyoxyalkylene alkylphenyl ethers, polyoxyalkylene phenylphenyl ethers, polyoxyalkylene alkyl esters, polyoxyalkylene alkyl esters, sorbitan fatty acid ester, polyoxyalkylene sorbitan alkyl esters, polyoxyalkylene sorbitan fatty acid esters, polyoxyalkylene sorbitan fatty acid esters, polyoxyalkylene glycerin fatty acid esters, polyglycerin alkyl ethers, polyglycerin fatty acid esters, sugar cane fatty acid esters, fatty acid alkanolamide, alkyl glycosides, polyoxyalkylene fatty acid bisphenyl ethers, polypropylene glycol, diethylene glycol, fluorine-based surface-active agents, polyoxyethylene/polyoxypropylene block polymer, and alkylpolyoxyethylene/polyoxypropylene block polymer ether.

The silicone-based surface active agents are typically represented by polyoxyalkylene-modified silicone, polyglyceryl-modified silicone, glyceryl-modified silicone, sugar-modified silicone, polyoxyethylene/polyoxypropylene block polymer, and alkylpolyoxyethylene/polyoxypropylene block polymer. Preferable silicone-based surface-active agents are exemplified by organopolysiloxane modified with linear-chain polyoxyalkylene (polyether-modified silicone having polyoxyalkylene groups bonded to side molecular chains and or to molecular terminals), copolymer of dimethylpolysiloxane and block-copolymer-type polyoxyalkylene, and linear-chain polyoxyalkylene/alkyl-modified organopolysiloxane (alkyl/polyether-modified silicone having polyoxyalkylene groups and alkyl groups bonded to side molecular chains and/or to molecular terminals). Also recommended for use are specific elastomer silicone polyethers (which are commercially available from US Dow Corning Co., Inc. as DC9011 Silicone Elastomer Blend) described in Japanese Patent No. 4080597 (Kokai H11-49957 and Kokai 2001-011281).

The amphoteric surface-active agents include imidazoline-type, amidobetaine-type, alkylbetaine-type, alkylamindobetaine-type, alkylsulfobetaine-type, amidosulfobetaine-type, hydroxysulfobetaine-type, carbobetaine-type, phosphobetaine-type, amidocarboxylic-acid type, and amidoamino-acid type amphoteric surface-active agents. Specific examples are the following: 2-undecyl-N,N,N-(hydroxyethyl carboxymethyl)-2-imidazoline sodium, 2-cocoyl-2-imidazolinium hydroxide-1-carboxyethyloxy-2 sodium salt, or a similar imidazoline-type surface-active agent; betaine lauryldimethyl amino acetate, myristyl betaine, or a similar alkyl betaine type surface-active agent; coconut oil fatty acid amidopropyl dimethylamino acetic acid betaine, palm kernel oil fatty acid amidopropyl dimethyl amino acetic acid betaine, tallow fatty acid amidopropyl dimethylamino acetic acid betaine, hydrogenated tallow fatty acidamidopropyl dimethylamino acetic acid betaine, amidopropyl dimethylamino acetic acid betaine laurate, amidopropyl dimethylamino acetic acid betaine myristate, amidopropyl dimethylamino acetic acid betaine palmitate, amidopropyl dimethylamino acetic acid betaine stearate, amidopropyl dimethylamino acetic acid betaine oleate, or a similar amidobetaine-type amphoteric surface-active agent; coconut oil fatty acid dimethylsulfopropyl betaine or a similar alkylsulfobetaine-type amphoteric surface-active agent; lauroyl dimethylamino hydroxysulfobetaine, or a similar alkylhydroxy sulfobetaine-type surface-active agent; laurylhydroxy phosphobetaine, or a similar phosphobetaine-type surface-active agent; N-lauroyl-N′-hydroxyethyl-N′-carboxymethyl ethylenediamine sodium, N-oleyl-N′-hydroxyethyl-N′-carboxymethyl ethylenediamine sodium, N-cocoyl-N′-hydroxyethyl-N′-carboxymethyl ethylenediamine sodium, N-lauroyl-N′-hydroxyethyl-N′-carboxymethylethylene diamine potassium, N-lauroyl-N′-hydroxyethyl-N′-carboxymethyl ethylenediamine potassium, N-lauroyl-N-hydroxyethyl-N′-carboxymethyl ethylenediamine sodium, N-oleoyl-N-hydroxyethyl-N′-carboxymethyl ethylenediamine sodium, N-cocoyl-N-hydroxyethyl-N′-carboxymethyl ethylenediamine sodium, N-lauroyl-N-hydroxyethyl-N′,N′-dicarboxymethyl ethylenediamine monosodium, N-oleoyl-N-hydroxyethyl-N′,N′-dicarboxymethyl ethylenediamine sodium, N-cocoyl-N-hydroxyethyl-N′,N′-dicarboxymethyl ethylenediamine monosodium, N-lauroyl-N-hydroxyethyl-N′,N′-dicarboxymethyl ethylenediamine disodium, N-oleoyl-N-hydroxyethyl-N′,N′-dicarboxymethyl ethylenediamine disodium, N-cocoyl-N-hydroxyethyl-N′,N′-dicarboxymethyl ethylenediamine disodium, or a similar amidoamino-acid type amphoteric surface-active agent.

The semi-polar surface-active agents include alkylamineoxide type surface-active agents, alkylamine oxides, alkylamide amine oxides, alkylhydroxyamine oxides, or the like. It is preferable to use alkyldimethylamine oxides having 10 to 18 carbon atoms, alkoxyethyl dihydroxyethylamine oxides, etc. Specific examples are the following: dodecyl dimethylamine oxide, dimethyl octyl amine oxide, diethyl decyl amine oxide, bis-(2-hydroxyethyl)dodecyl amine oxide, dipropyl tetradecyl amine oxide, methyl ethyl hexadecyl amine oxide, dodecyl amide propyl dimethylamine oxide, cetyl dimethylamine oxide, stearyl dimethylamine oxide, tallow dimethylamine oxide, dimethyl-2-hydroxyoctadecyl amine oxide, lauryl dimethylamine oxide, myristyl dimethylamine oxide, stearyl dimethylamine oxide, isostearyl dimethylamine oxide, coconut fatty acid alkyl dimethylamine oxide, amidopropyl dimethylamine oxide caprylate, amidopropyl dimethylamine oxide laurate, amidopropyl dimethylamine oxide myristate, amidopropyl dimethylamine oxide palmitate, amidopropyl dimethylamine oxide stearate, amidopropyl dimethylamine oxide isostearate, amidopropyl dimethylamine oxide oleate, amidopropyl dimethylamine oxide 12-hydroxystearate, coconate fatty acid amidopropyl dimethylamine oxide, palm-kernel oil fatty acid amidopropyl dimethylamine oxide, castor oil fatty acid amidopropyl dimethylamine oxide, amidoethyl dimethylamine oxide laurate, amidoethyl dimethylamine oxide myristate, coconut fatty amidoethyl dimethylamine oxide, amidoethyl diethylamine oxide laurate, amidoethyl diethylamine oxide myristate, coconut fatty acid amidoethyl diethylamine oxide, amidoethyl dihydroxyethylamine oxide myristate, and coconut fatty acid amidoethyl dihydroxyethylamine oxide.

Component (G) is a film-forming agent that comprises a high-molecular component for forming film structures on skin and hair. Such film-forming agents may be selected from non-ionic polymers, cationic polymers, anionic polymers, and aliphatic polymers, which are listed below. Component (G) may be composed of one or more non-ionic polymers, or from two or more of polymers of different types. Although the aforementioned film-forming agents are not necessarily water-soluble compounds, for thickening of water-containing components it is recommended to add water-soluble polymer (H) or incorporate this polymer into the film-forming agent.

Favorable nonionic polymers include polyvinyl pyrrolidone (Rubiskol K, the product of BASF Corporation), vinyl pyrrolidone/vinyl acetate copolymers (Rubiskol VS, the product of BASF Corporation), vinyl pyrrolidone/dimethylaminoethyl methacrylate copolymer (Copolymer—937, the product of ISP Corporation), vinyl caprolactam/vinyl pyrrolidone/dimethylaminoethyl methacrylate copolymer (Copolymer—VC713, ISP Corporation), polyvinyl alcohol, polyoxylpropylene butyl ether, or a similar nonionic polymer compound.

Favorable cationic polymers include, e.g., a quaternary compound of vinylpyrrolidone/dimethylaminoethyl methacrylate (GAFQUAT, the product of ISP Corporation), a methylvinyl imidazolium chloride/vinyl pyrrolidone copolymer (RubiCoat, the product of BASF Corporation), cationized cellulose, cationized starch, cationized guar gum, diethyl sulfate of vinyl pyrrolidone-N,N-dimethylaminoethyl methacrylic acid copolymer, a polymer of a diallyl aminoethyl quaternary ammonium salt, or a similar cationic polymer compound.

Favorable anionic polymers include, e.g., an ester acrylate/ester methacrylate copolymer (Plus-Size®, the product of Goo Chemical Co., Inc.), vinyl acetate/crotonic acid copolymer (Resyn 28-1310®, NSC Corporation), vinyl acetate/crotonic acid/vinyl neodecanoate copolymer (Resyn 28-2913®, NSC Corporation), methylvinyl ether maleic acid half-ester (Gantrez ES®, the product of ISP Corporation), t-butylacrylate/ethyl acrylate/methacrylic acid copolymer (Luvimer®, the product of BASF Corporation), vinyl pyrrolidone/vinylacetate/vinyl propionate copolymer (Luviskol YAP®, the product of BASF Corporation), vinyl acetate/crotonic acid copolymer (Luviset CA®, the product of BASF Corporation), vinyl acetate/crotonic acid/vinyl pyrrolidone copolymer (Luviset CAP®, the product of BASF Corporation), vinyl pyrrolidone/acrylate copolymer (Rubiflex, the product of BASF Corporation), acrylate/acrylamide copolymer (Ultrahold®, the product of BASF Corporation), vinylacetate/butyl maleate/isobornyl acrylate copolymer (Advantage®, the product of ISP Corporation), acryl resin alkanolamine, an urethane-modified acryl-based polymer described in International patent Application Publication WO2008/004502, and other anionic polymer compounds.

Favorable amphoteric polymers include, e.g., acetic acid amphoteric compounds of dialkylaminoethyl methacrylate (Ukaformer®, the product of Mitsubishi Petrochemical Co., Ltd.), octylacrylamine acrylate/hydroxypropyl acrylate/butyl aminoethyl methacrylate copolymer (Amphomer, the product of NSC Corporation), octylacrylamide-butylaminoethyl methacrylate-acrylate copolymer, or similar amphoteric polymers.

Component (H) is a water-soluble polymer that is added to a cosmetic material for improving its sense of use. When this component is used in conjunction with a conventional cosmetic, it may be in any form, such as an amphoteric, cationic, anionic, nonionic, or a water-swelling clay-mineral form. The aforementioned water-soluble polymers may be used individually or in combinations of two or more. Since these water-soluble polymers produce a thickening affect on water-containing components, they are especially advantageous for use with gel-like water-containing cosmetic materials, water-in-oil type emulsion cosmetics, and oil-in-water type emulsion cosmetics.

The amphoteric water-soluble polymers include amphoteric starch, derivatives of a dimethyl diallyl ammonium chloride (e.g., an acrylamide/dimethyldiallyl ammonium copolymer and an acrylic acid/dimethyl diallyl ammonium chloride copolymer), derivative of methacrylic acid (e.g., polymethacryloyl ethyl dimethyl bentaine, N-methacryloyloxyethyl N,N-dimethylammonium-α-methyl carboxy betaine/alkyl methacrylate copolymer, etc.).

The cationic water-soluble polymers include quaternary nitrogen-modified polysaccharides (e.g., cation-modified cellulose, cation-modified hydroxyethyl cellulose, cation-modified guar gum, cation-modified locust bean gum, cation-modified starch, etc.), derivatives of dimethyl diallyl ammonium chloride (such as dimethyl diallyl ammonium chloride/acrylamide copolymer, dimethyl methylene piperidinium polychloride, etc.), vinyl pyrrolidone derivatives (such as vinyl pyrrolidone/dimethylamino ethyl methacrylic acid copolymer salt, vinyl pyrrolidone/methacrylamide propyl trimethyl ammonium chloride copolymer, vinyl pyrrolidone/methyl vinylimidazolium chloride copolymer, etc.), methacrylic acid derivatives (e.g., methacryloyl ethyl dimethyl betaine/methacryloyl; ethyl trimethyl ammonium chloride/methacrylic acid 2-hydroxyethyl copolymer, and methacryloyl dimethyl betaine/methacryloyl ethyl trimethyl ammonium chloride/methacryloxy polyethyleneglycol methacrylate copolymer, etc.).

Anionic water-soluble polymers include polyacrylic acid or alkali metal salts of polyacrylic acid, polymethacrylic acid or alkali metal salts of polymethacrylic acid, hyaluronic acid or alkali metal salts of hyaluronic acid, acetylated hyaluronic acid or alkali salts of acetylated hyaluronic acid, hydrogenated products of methyl vinyl ether/anhydrous maleic acid copolymer, or similar water-soluble polymers of aliphatic carboxylic acids or metal salts of these acids, carboxymethyl cellulose or alkali metal salts of carboxymethyl cellulose, methylvinylether-maleic acid-half ester copolymer, acrylic resin alkanoyl amine solutions, and carboxyvinyl polymers.

Nonionic water-soluble polymers include polyvinyl pyrrolidone, high-molecular-weight polyethyleneglycol, vinyl pyrrolidone/vinyl acetate copolymer, vinyl pyrrolidone/dimethylaminoethyl methacrylate copolymer, vinyl caprolactam/vinyl pyrrolidone/dimethylaminoethyl methacrylate copolymer, cellulose or cellulose derivatives (such as methyl cellulose, ethyl cellulose, hydroxyethyl cellulose, hydroxypropyl cellulose, carboxymethyl cellulose), keratin, collagen or derivatives thereof, calcium alginate, pullulan, agar-agar, gelatin, tamarind seed polysaccharides, xanthan gum, carrageenan, high-methoxyl pectin, low-methoxyl pectin, guar gum, pectin, gum arabia, crystalline cellulose, arabinogalactan, karaya gum, gum tragacanth, alginic acid, albumin, casein, curdlan, guran gum, starch, quince seed gum, gum tragacanth, chitin, chitosan derivatives, starch (rice, corn, potato, wheat, etc.), keratin, collagen, collagen derivatives, and other natural polymer compounds.

The water-swelling clay minerals are inorganic water-soluble polymers. This mineral can be exemplified by one type of colloid-containing aluminum silicate that has a three-layer structure and that is represented by the following formula (1):

(X,Y)₂₋₃(Si,Al)₄O₁₀(OH)₂Z_{1/3}.nH₂O (1)

(where, X is Al, Fe(III), Mn(III), or Cr(III); Y is Mg, Fe(II), Ni, Zn, or Li; and Z is K, Na, or Ca).

The aforementioned water-swelling clay minerals may be exemplified by the following substances: bentonite, montmorillonite, pyderite, nontronite, saponite, hectorite, aluminum magnesium silicate, and silicic acid anhydride. These minerals may be natural or synthesized.

Component (I) is a UV blocker that may comprise an organic-based component or inorganic-based component having a property of UV absorption or protection. It is recommended to combine organic and inorganic UV protective agents, in particular to combine UV protective agents against UV-A radiation with UV protective agents against UV-B radiation.

The inorganic UV blocking components may comprise inorganic powdered inhibitors that include the inorganic coloring agent, powdered metal pigments, etc. that were mentioned earlier as aforementioned component (C), titanium oxide, zinc oxide, cerium oxide, lower-valent titanium oxide, iron-doped titanium oxide, iron hydroxide, or a similar metal hydroxide, plate-like oxide particles, aluminum flakes, or similar metal flakes, silicon carbide, or similar ceramics. Among these, it is recommended to choose at least one type of super-fine metal oxides or metal hydroxides with an average particle diameter in the range of 1 to 100 nm.

The organic UV blocking components may be exemplified by the following substances: homomethyl salicilate, octyl salicilate, triethanolamine salicilate, or a similar salicylic acid based substance; para-aminobenzoic acid, ethyl dihydroxypropyl para-aminobenzoic acid, glyceryl para-aminobenzoic acid, octyldimethyl para-aminobenzoic acid, paradimethylaminobenzoic acid amyl, paradimethylaminobenzoic acid 2-ethylhexyl, or similar PABA-based substances; 4-(2-β-glucopyranosyloxy)propoxy-2-hydroxy benzophenone, dihydroxydimethoxy benzophenone, dihydroxydimethoxy benzophenone sodium disulfonate, 2-hydroxy-4-methoxy benzophenone, hydroxymethoxy benzophenone sulfonic acid or trihydrates thereof, hydroxymethoxy benzophenone sodium sulfonate, 2-hydroxy-4-methoxy benzophenone-5-sulfuric acid, 2,2′-dihydroxy-4-methoxybenzophenone, 2,2′-dihydroxy-4-methoxybenzophenone, 2,2′-dihydroxy-4,4′-dimethoxybenzophenone, or similar benzophenone-based substances; para-methoxy cinnamic acid 2-ethylhexyl (also known as octyl para-methoxy cinnamate), di-para-methoxycinnamic acid mono-2-ethyl hexanoic acid glyceryl, 5-diisopropyl cinnamic acid methyl, 2,4,6-tris[4-(2-ethylhexyloxycarbonyl)aniline]-1,3,5-triazine, trimethoxy cinnamic acid methyl bis(trimethylsiloxy)silylisopentyl, a mixture of para-methoxy cinnamic acid isopropyl and diisopropyl cinnamic acid ester, p-methoxy hydrocinnamic acid diethanolamine salt, or similar cinnamic-acid-based substances; 2-phenyl-benzoimidazole-5-sulfuric acid, 4-isopropyl dibenzoyl methane, 4-tert-butyl-4′-methoxy dibenzoyl methane, or similar benzoyl-methane-based substances; 2-cyano-3,3-diphenylprop-3-ane acid 2-ethylhexyl ester (also known as octocrylene), dimethoxybenzylidene dioxoimidazolidine propionic acid 2-ethylhexyl, 1-(3,4-dimethoxyphenyl)-4,4-dimethyl-1,3-pentadiol, cinoxate, methyl-O-aminobenzoate, 2-ethylhexyl-2-cyano-3,3-diphenyl acrylate, 3-(4-benzylidene)camphor, octyl triazone, 4-)3,4-dimethoxy phenyl methylene)-2,5-dioxyno-1-imidazolidine propionic acid 2-ethylhexyl, and polymer derivatives and silane derivatives thereof.

The aforementioned organic UV blocking components may be added in a form of encapsulated in the polymer powder. The polymer powder may be hollow or non-hollow, with average particle diameter in the range of 0.1 to 50 μm, and with broad or sharp particle size distribution. Polymer types may be exemplified by acrylic resins, methacrylic resins, styrene resins, polyurethane resins, polyethylene, polypropylene, polyethylene terephthalate, silicone resins, Nylon, and acrylamide resins. It is recommended that polymer powder contain 1 to 30 mass % of the organic UV blocking component, in particular such a UV-A blocker as 4-tert-butyl-4′-methoxy dibenzoyl methane.

Component (J) is a lower alcohol that is used as a solvent, solubilization agent, or dispersion medium for components of various cosmetic raw materials. It is recommended to add lower alcohols to detergents, bactericides, astringents, drying accelerators, etc., and, in particular, to cosmetic materials for which the sense of fresh touch is an important factor. Examples of lower alcohols include isopropanol, n-propanol, t-butanol, s-butanol, etc., of which ethanol is the most favorable.

Component (K) is represented by polyhydric alcohols, which, similar to lower alcohols, can be used as solvents for various components of cosmetic raw materials, and in addition have a moisture-retaining effect, and therefore, is used for improving the sense of moist touch. More specifically, component (K) can be exemplified by the following compounds: 1,3-butylene glycol, 1,2-butylene glycol, propylene glycol, trimethylene glycol, tetramethylene glycol, 2,3-butylene glycol, pentamethylene glycol, 2-butene-1,4-diol, dibutylene glycol, pentyl glycol, hexylene glycol, octylene glycol, or a similar dihydric alcohol, glycerin, trimethylolpropane, 1,2,6-hexanetriol, or similar trihydric alcohols, pentaerythritol, xylitol, or other tetrahydric or higher hydricity alcohols, sorbitol, mannitol, maltitol, maltrose, sucrose, erythritol, glucose, fructose, starch decomposition products, maltose, xylitose, starch sugar reduced alcohol, or a similar sugar alcohol. In addition to lower polyhydric alcohols, polyhydric alcohol polymers, such as diethylene glycol, dipropylene glycol, triethylene glycol, polypropylene glycol, tetraethylene glycol, diglycerin, polyethylene glycol, triglycerin, tetraglycerin, polyglycerin, etc., also can be used.

Component (K) may be exemplified by 3-butylene glycol, sorbitol, dipropylene glycol, glycerin, polyethylene glycol, etc. In particular, polyhydric alcohols that may be selected for improving the moisture-retention affect of cosmetic materials can be selected from 1,3-butylene glycol, sorbitol, dipropylene glycol, glycerin, and polyethylene glycol.

Water (L) should not contain health-hazardous components, should be clean, and may comprise tap water, purified water, ion-exchange water, or mineral water. Since compositions with high concentration of lower alcohol, such as ethanol that may constitute component (J), are highly flammable, addition of water will reduced their point of flammability and will improve safety during manufacture, storage, and transportation. Furthermore, in preparation of cosmetic compositions, the water-soluble components, such as water-soluble ionic surface-active agents, can be preliminarily dispersed in water.

There are no special restrictions with regard to the amount in which water is to be added to the cosmetic material of the invention. It is recommended, however, to add water, i.e., component (L), in an amount of 0.1 to 4,000 parts by mass, preferably, 5 to 2,000 parts by mass, per 100 parts by mass of the sum of the phenyl-containing organopolysiloxane composition and oil agent (D) that contains aforementioned cosmetic components. By using the aforementioned surface-active agents, the obtained cosmetic material can be prepared as W/O emulsion, O/W emulsion, etc.

In addition to silicone resin of component (A), the cosmetic material of the invention may also include at least one type of another substance selected from an acryl silicone resin, an acryl silicone dendrimer copolymer, polyamide-modified silicone, acryl-modified silicone resin wax, or an organic surface-active agent.

It is recommended that the silicone comprise a solid compound having a silicone network structure and be represented by TDQ resin, TQ resin, TD resin, MDTQ resin, MTQ resin, MDQ resin, MQ resin composed of arbitrary combinations of tetra-functional siloxy units (Q units), monoalkylsiloxy units (T units), dialkylsiloxy units (D units), or trialkylsiloxy units (M units). Substituents bonded to silicon atoms of these resins may comprise not [merely] alkyl groups but also substituted alkyl groups. Among these compounds, the fluorine-modified silicone resin, trimethylsiloxy silicic acid (MQ resin), and dimethylsiloxy-containing trimethylsiloxy silicic acid (MDQ resin) are preferable since they impart excellent sense of use.

The acryl silicone resin of an acryl/silicone graft- or block-copolymer type is also suitable for use. Also useful is an acryl silicone resin that contains in its molecule a portion that constitutes a pyrrolidone part, a long-chain alkyl part, a polyoxyalkylene part, a fluoroalkyl part, a carboxylic-acid part, or a similar anionic part.

A specific example of an especially favorable acryl silicone dendrimer copolymer is a vinyl-based polymer that has a carbosiloxane-dendrimer structure in its side molecular chain (see Kokai 2000-063225). This compound is commercially produced by Dow Corning Toray Co., Ltd. as FA 4001 CM Silicone Acrylate, FA 4002 ID Silicone Acrylate, etc.

The polyamide-modified silicone can be exemplified by the siloxane-based polyamide described in U.S. Pat. No. 5,981,680 and is commercially produced by American Dow Corning Corporation as 2-8178 Gellant, 2-8179 Gellant, etc.

The alkyl-modified silicone resin wax may be exemplified, e.g., by the silsesquioxane resin wax described in Japanese Publication No. 2007-532754.

Depending on the function of the cosmetic material, the latter can be combined at least with one type of the compounds selected from amino acid or its salts, inorganic salts, and organic acid it its salts.

The amino acid and/or salts thereof can be exemplified by valine, leucine, isoleucine, serine, threonine, phenylalanine, arginine, lysine, aspartic acid, glutamic acid, cystine, cysteine, methionine, and tryptophan.

The inorganic salts may be exemplified by alkali-metal salts, alkali earth metal salts, aluminum salts, zinc salts, or ammonium salts of hydrochloric acid, sulfuric acid, nitric acid, carbonic acid, phosphoric acid, etc. Preferable inorganic salts are sodium salts, potassium salts, magnesium salts, calcium salts, aluminum salts, zinc salts, ammonium chloride, or other chlorides, sodium sulfate, potassium sulfate, magnesium sulfate, aluminum sulfate, zinc sulfate, ammonium sulfate, or other sulfates, sodium nitrate, potassium nitrate, magnesium nitrate, calcium nitrate, aluminum nitrate, zinc nitrate, ammonium nitrate, or other nitrates, sodium carbonate, potassium carbonate, magnesium carbonate, calcium carbonate, or other carbonates, sodium phosphate, potassium phosphate, or other phosphates. Among these, most preferable are sodium chloride, potassium chloride, magnesium chloride, calcium chloride, sodium sulfate, potassium sulfate, magnesium sulfate, and aluminum sulfate.

The organic acids can be exemplified by acetic acid, lactic acid, citric acid, ascorbic acid, maleic acid, tartaric acid, or a similar acid. The organic acid salts can be represented by sodium acetate, potassium acetate, sodium ascorbate, as well as by sodium citrate, sodium lactate, sodium glycolate, sodium maleate, sodium tartarate, or a similar α-hydroxylic acid salt, sodium aspartate, potassium aspartate, magnesium aspartate, calcium aspartate, sodium glutamate, potassium glutamate, magnesium glutamate, calcium glutamate, arginine-glutamate, ornithine-glutamic acid salt, lysine-glutamic acid salt, lysine-aspartic acid salt, or a similar amino acid salt, sodium alginate, etc.

The cosmetic product of the invention may further be combined with biologically active substances, such as plant extracts, or the like, pH adjusters, antioxidants, chelating agents, moisture retainers, fragrants, bactericidal and antiseptic agents, or similar components conventionally added to cosmetic materials. There are no special limitations with regard to these additives, provided they are not in conflict with the objects of the present invention.

The biologically active substances are those substances that impart some biological activity to the skin when applied to the latter. Examples of such substances are the following: anti-inflammatory agents, anti-aging agents, skin-lightening agents, pore-tightening agents, antioxidants, hair growing agents, hair tonics, blood circulation promoters, bactericides, antiseptics, desiccants, cool feeling agents, warm-sensation agents, vitamins, wound-healing accelerators, irritation relievers, pain relievers, cell activators, enzymes, etc. Most suitable for used may be one or several biologically active substance selected from the group consisting of anti-inflammatory agents, anti-aging agents, skin-lightening agent, hair growing agents, hair tonics, blood circulation promoter, bactericides, antiseptics, vitamins, wound-healing accelerators, irritation relievers, pain relievers, cell activators, and enzymes.

The aforementioned components may be exemplified by the following substances: angelica keiskei extract, avocado extract, sweet Hydrangea leaf extract, arnica extract, aloe extract, apricot extract, apricot kernel extract, gingko extract, fennel extract, oolong tea extract, rose fruit extract, Echinacea leaf extract, extract of scutellaria root, Phellodendron amurense extract, extract of Japanese coptis, barley extract, hypericum perforatum extract, Lamium album extract, Hypericum erectrum abstract, nasturtium officinale extract, orange extract, dried products of sea water, sea weeds, hydrogenated elastin, hydrolyzed wheat flour, hydrolyzed silk, chamomile extract, carrot extract, Artemisia capillaries extract, licorice extract, hibiscus flower extract, cinchona extract, cucumber extract, guanosine, gardenia florida extract, Sasa albo-marginata, essence, crataegus extract, walnut extract, grapefruit extract, clematis extract, chlorella extract, mulberry extract, gentian extract, black tea extract, yeast extract, arctium lappa root extract, rice bran fermented extract, oryza sativa (rice) germ oil, confrey extract, collagen, cowberry extract, extract of Asarum sieboldii, bupleurum extract, umbilical extract, salvia extract, soapwort extract, bamboo grass extract, crataegus extract, zanthoxylum extract, shiitake mushroom extract, extract of Rehmannia root, extract of lithospermum root, perilla extract, extract of Tilia japonica, spiraea ulmaria extract, peony extract, extract of calamus root, white birch extract, extract of Equisetum arvense L., extract of Hedera helix L., crataegus oxyacantha extract, extract of Sambucus nigra, Achillea milefolium extract, peppermint extract, sage extract, tree mallow extract, cnidium extract, extract of Swertia japonica, sialid extract, soybean extract, extract of fruits of common jujube, thyme extract, tea extract, clove extract, cogon extract, extract of dried orange peel, capsicum frutescens fruit extract, calendula extract, peach kernel extract, fruit skin of Citrus aurantium, bitter orange peel extract, extract of Houttuynia cordata, tomato extract, fermented soybean extract, ginseng extract, garlic extract, wild rose extract, hibiscus extract, extract of roots of Ophiopogon japonicus, lotus extract, parsley extract, honey, hamamelis extract, Parietaria extract, extract of Isodon japonicus, bisabolol, loquat extract, coltsfoot extract, butterbur flower extract, poria cocos extract, butcher's-broom extract, grape extract, propolis, luffa extract, safflower extract, extracts of peppermint, tilia platyphyllos flower extract, paeonia suffruticosa root extract, hop extract, pine extract, horse chestnut extract, extract of Sapindus mukurossi, Melissa extract, peach extract, centaurea cyanus flower extract, eucalyptus extract, extract of Saxifraga stolonifera, extract of Citrus junos, coix seed extract, artemisia princeps leaf extract, lavender extract, apple extract, lettuce extract, lemon extract, astragalus sinicus extract, rose extract, rosemary extract, Roman chamomile extract, royal jelly extract, etc.

In addition, the following substances can be used: desoxyribonucleic acid, mucopolysaccharide, sodium hyaluronate, sodium chondroitin sulfate, collagen, elastin, chitin, chitosan, hydrolyzed eggshell membrane, or similar biopolymers, estradiol, ethinyl estradiol, or similar hormones, sphingolipid, ceramide, cholesterol derivatives, phospholipid, and other oil components, ε-aminocaproic acid, glycyrrhizic acid, β-glycyrrhetic acid, lysozyme chloride, guaiazlene and hydrocortisone; lysozyme chloride, guaiazlene, hydrocortisone, allantoin, tranexamic acid, azylene, or other anti-inflammatory substances, vitamins A, B2, B6, C, D, E, potassium pantothenate, biotin, nicotinic acid amide, vitamin C ester, or other vitamins, allantoin, diisopropylamine dichloroacetate, 4-aminomethyl cyclohexane carboxylic acid, or other active components, carotenoid, flavonoid, tannin, lignin, saponin, and other antioxidants, α-hydroxy acid, β-hydroxy acid, or other cell activators, γ-orizanol, vitamin E derivatives, or other blood circulation promoters, retinal, retinol derivatives, or other wound healing agents, sefarantin, liquorice root extract, capsicum tincture, hinokitiol, sulfurized garlic extract, pyridoxine chloride, DL-α-tocopherol, DL-α-tocopheryl acetate, nicotinic acid, nicotinic acid derivatives, calcium pantothenate, D-pantothenyl alcohol, acetyl pantothenylethyl ether, allantoin, isopropylmethylphenol, capronium chloride, benzalkonium chloride, diphenhydramine chloride, Takanal, vanillylamide nonylate, vanillylamide nonanoate, pyroctone olamine, glyceryl pentadecanoate, L-menthol, camphor, or other refrigerants, mononitroguaiacol, resorcinol, γ-aminobutyric acid, benzethonium chloride, mexiletine hydrochloride, auxin, female hormone, cantharis tincture, cyclosporine, zinc pyrithione, hydrocortisone, minoxidil, polyoxyethylene sorbitan monostearate, peppermint oil and sasanishiki [Japanese rice] extract, or similar and hair growing agents.

The skin-beautifying components include, e.g., hydroquinone, placental extract, arbutin, glutathione, saxifraga sarementosa extract, or other skin-whitening agents, royal jelly, or other cell activating agents, skin ameliorating agents, nonylic acid vanillylamide, nicotinic acid benzyl ester, nicotinic acid β-butoxy ethyl ester, capsaicin, zingerone, cantharis tincture, ichthammol, caffeine, tannic acid, α-borneol, tocopherol nicotinate, inositol hexanicotinate, cyclandelate, cinnarizine, tolazoline, acetylcholine, verapamil, cepharanthine, γ-orizanol, and other blood-circulation promoters, zinc oxide, tannic acid, or similar skin astringent agents, sulfur, thiantol, and other anti-seborrheic agents, vitamins such as vitamin A oil, retinol, retinol acetate, retinal palmitate, or a similar group A vitamin, riboflavin, riboflavin acetate, flavin adenine dinucleotide, or another group B2 vitamin, pyridoxine hydrochloride, pyridoxine dioctanoate, pyridoxine tripalmitate, or another group B6 vitamin, vitamin B12 or derivatives thereof, vitamin B15 or derivatives thereof, or other vitamins of group B, L-ascorbic acid, L-ascorbic acid dipalmitic acid ester, L-ascorbic acid 2-sulfate, L-ascorbic acid phosphoric acid diester dicalcium, or a similar group C vitamin, ergocalciferol, cholecalciferol, or similar group D vinamin, α-tocopherol, β-tocopherol, γ-tocopherol, dL-α-tocopherol acetate, DL-α-tocopherol nicotinate, DL-α-tocopherol succinate, or similar group E vitamin, vitamin H, vitamin P, nicotinic acid, benzyl nicotinate, or a similar nicotinic acid-type compound, calcium pantothenate, D-pantothenyl alcohol, pantothenyl ethyl ether, acetylpentothenyl ethyl ether, or a similar pantothenyl acid-type compound.

The pH adjuster may be exemplified, e.g., by lactic acid, citric acid, glycolic acid, siccinic acid, tartaric acid, DL-maleic acid, potassium carbonate, sodium hydrocarbonate, ammonium bicarbonate, etc. These pH adjusters may be the same as or different from acid bases used for the preparation of cationic surface-active agents from aforementioned tertiary amines and amidoamines.

The chelating agents are used for rendering mineral ions insoluble in water. Such chelating agents can be exemplified by EDTA, alanine, sodium edatate, sodium polyphosphate, sodium metaphosphate, and phosphoric acid.

The antioxidant can be exemplified by tocopherol, butyl hydroxytoluene, dibutyl hydroxytoluene, phytic acid, etc.

The moisture retainers may be represented by hyaluronic acid, chondroitin sulfuric acid, pyrrolidone carboxylate, polyoxyethylene methyl glycoside, polyoxypropylene methyl glucoside, etc.

The fragrants are added to cosmetics for imparting to them aroma and perfume, as well as for masking ill smells. There are no special limitation with regard to conventional fragrants that can be added to the cosmetic materials, and these fragrants may include various extracts exemplified earlier for physiologically active components, as well as fragrants obtained by extraction from various flowers, seeds, leaves, roots, and seaweeds, fragrants extracted from various parts of animal bodies such as musk, whale sperm), and artificially synthesized fragrants (such as menthol, musk, ester acetate, vanilla).

The bactericides and antiseptics may be represented, e.g., by paraoxybenzoic acid alkyl ester, benzoic acid, sodium benzoate, sorbitic acid, potassium sorbitate, phenoxyethanol, etc. The antiseptics can be exemplified by benzoic acid, salicylic acid, carbolic acid, sorbitic acid, paraoxybenzoic acid ester, parachlorometacresol, hexachlorophene, chlorobenzalconium, chlorohexidine, trichlorocarbanilido, sensitizing dye and light-sensitive elements. However, these substances are not applicable to lipstick.

Cosmetic products that relate to the present invention are characterized by including the aforementioned phenyl-containing organopolysiloxane composition and other appropriate components. Specific examples of these cosmetic products are the following: skin cleansing products, skin care products, makeup products, antiperspirant products, UV blockers, and other skin-related cosmetics; hair-washing agents, hair-dressing products, hair-coloring products, hair-growing products, hair-rinsing products, hair-treatment products, and other head hair cosmetics; hair-growing agents, hair tonics, paint-relieving agents, bactericidal agents, anti-inflammatory agents, refrigerants, and skin anti-ageing agents. However, this list should not be construed as limitative.

The aforementioned skin cosmetics may also be in the form of A cosmetic material for head skin, face skin (including lips, eyebrows, and cheeks), fingers, nails, and the entire body. Specific examples are the following: cleansing gels, cleansing creams, cleansing foams, facial creams, eye makeup removers, facial foams, liquid soaps (body soaps), hand soaps, gel soaps, shaving creams, nail polish remover, anti-acne cosmetics, and other skin-cleansing cosmetic products; body creams, head treatments, skin milks, milk lotion, emulsions, facial packs, body powders, essences, shaving lotions, massage cosmetics, and other skin-care products; foundations, liquid foundations, oily foundations, makeup bases, white powders, face powders, blushes, lip glosses, eye cream, mascara, eyebrow cosmetics, eyelashes cosmetics, or other makeup products; deodorants, or other perspiration deodorants; sun screens, sun tanning substances, or other UV radiation controlling products.

The aforementioned head hair cosmetics include shampoo, rinsing shampoo, or a similar hair washing substances; hair wax, hair curl holders, hair setting agents, hair creams, hair sprays, hair liquids, or other hair dressing cosmetics; hair dyes, hair color sprays, hair color rinses, hair color sticks, or other hair coloring cosmetic products; hair tonics, hair treatment essences, hair packs, or other hair growth simulating agents; oil rinses, cream rinses, treatment rinses, hair conditioners, and hair treatment, or other hair rinses or hair conditioners.

In particular, the cosmetic products of the present invention that include the aforementioned phenyl-containing organopolysiloxane composition are preferable for use as a makeup cosmetic, and especially as a face makeup cosmetic for lips, eyelashes, and area around the eyes. When the makeup cosmetic of the invention is used in the areas mentioned above, it continuously suppress elution of sweat and sebum, prevents deterioration of the cosmetic, and at the same time creates the sense of transparency, brightness and gloss. Such cosmetics include lip creams, kneaded paste of lip-crimson, lip glosses, lipsticks, lip liners, or other lip cosmetics; eye shadows, mascaras, eye liners, eye colors, or other eye makeup cosmetics.

There are no special restrictions with regard to the form in which the cosmetic materials of the invention can be used, and the cosmetics can be produced in the form of a liquid, W/O emulsion, O/W emulsion, W/O cream, O/W cream, solid substance, paste, gel, powder, laminated product, moose, mist, granules, flakes, crushed substance, etc. Among these, most preferable are W/O creams, solids, pastes, gels, and powders.

The makeup cosmetic material of the present invention can be obtained by the method described below. When an eye makeup cosmetic material has to be prepared as an O/W emulsion, it is recommended to use among the aforementioned cosmetic raw materials those that allow to adjust an oil phase consisting of the phenyl-containing organopolysiloxane composition and an oil agent (D), etc., and to emulsify the obtained oil phase by applying a mechanical force in an aqueous phase obtained by mixing water (L) with hydrophilic raw materials. In the case of an oil type, make-up cosmetic products can be obtained by mixing components of various raw materials with heating.

In the case of an oil-based lip cosmetic, first an oil phase is prepared by heating and kneading a mixture obtained from the phenyl-containing organopolysiloxane composition of the invention and oil agent (D), and then powder (C) or coloring components, etc. are dispersed in the obtained oil phase, and the obtained product is loaded into a cosmetic container. In the case of a water-based lip cosmetic, first an oil phase is prepared by heating and kneading a mixture obtained from the phenyl-containing organopolysiloxane composition of the invention and oil agent (D), then an aqueous phase is prepared by dispersing a cosmetic raw material that contains water (L), surface-active agent, and powders or colorants, and emulsifying the oil phase in the water phase by applying a mechanical force.

There are no special restrictions with regard to the type of a cosmetic container into which the cosmetic material of the invention can be loaded, and the container can be made in the form of a jar, pump, tube, bottle, dispensing-port container, pressure-proof aerosol container, light-shielding container, compact container, metal-tray type container, stick container, feeding container, spray container, partitioned container with a mixing dispensing port, etc.

### EXAMPLES

The invention will be further described by way of application examples, which, however, should not be construed as limitative. In these examples, all amounts in the table are given in mass % units, and the numbers in prescription examples are also given in mass % units. The methods of measuring physical properties of various components and methods of evaluation of characteristics of components contained in Application Examples and in Comparative Examples are described below.

[Evaluation of the “Appearance” of Organopolysiloxane Composition]

The organopolysiloxane compositions having constituents shown in Table 1 (Samples Nos. 1 to 9) were loaded into glass containers and visually evaluated based on the criteria given below. Since symbol “X” designates separation of components, further evaluation was not conducted.
◯: the entire composition is uniformly transparent;
Δ: turbulence is observed in the composition, but the entire composition is uniform
X: all components are separated, the composition is obtained in a non-uniform state.

[Viscosity of Organopolysiloxane Composition]

Viscosities of the organopolysiloxane compositions shown in Table 1 ((Samples Nos. 1 to 9) were measured at 25° C. by a capillary tube viscosity measurement method with the use of an Ubbelohde viscometer. All the viscosities higher than 30,000 mPa·s, were evaluated as [>30,000].

[Measurement of Index of Refraction (RI)]

The indices of refraction of various components and of the organopolysiloxane compositions shown in Table 1 (Samples Nos. 1 to 9) were measured by means of an Abbe refractometer.

[Measurement of Number-Average Molecular Weight (Mₙ) of Silicone Resin]

The number-average molecular weights (Mₙ) shown in Application and Comparative Examples were determined by a calibration curve method under measurement conditions shown below and on the results of gel chromatography analysis, and the measured values of the number-average molecular weights were recalculated to a polystyrene standard.

### <Measurement Conditions>

Measurement Instrument: the product of Waters Co., Inc.; GPC measurement instrument equipped with RI detection device (2695)
Column: PL gel MIXED-C column (the product of Polymer Laboratories Co., Ltd.; 300 mm) C×2 (connected in series)

### Measurement Temperature: 40° C.

Flow Rate: 1 mL/min
Solvent: tetrahydrofuran (THF)
Concentration of sample: 0.5 mass % in THF solution
Reference Substance: polystyrene

### Prescription Example 1

### Evaluated as a Transparent Lipstick

The following evaluation criteria were used for making a transparent lipstick from the organopolysiloxane composition prepared in Prescription Example 1 described below:
◯: the entire product is transparent, and appearance with glitter remains for more than 1 months;
X: the product produces the sense of non-uniform transparency, and separation of components was observed within a month.

### Prescription Example 3

### Evaluated as a Blush

The following evaluation criteria were used for making a transparent lipstick from the organopolysiloxane composition prepared in Prescription Example 1 described below:
◯: the entire product is obtained in the form of a uniform paste with glitter; this condition remains stable for more than 1 months;
X: the oil phase is separated in a month; non-uniformities are observed in both the oil layer and the paste layer over the entire product.

[Silicone Resin]

Silicone resins (A-1), (A-2), (CA-1 to CA-3) having the following structures, contents of phenyl, and number-average molecular weights (Mₙ) (Samples 1 to 9) were used for the preparation of the organopolysiloxanes shown in Table 1:
silicone resin (A-1): phenylsilsequioxane that consists essentially of T^{Ph} units
(Mₙ: 1500; phenyl group content: 60 mass %; hydroxyl group content: 6 mass %; flake-like at room temperature);
silicone resin (A-2): phenylsilsequioxane that consists of propylsiloxane units (C₃H₇SiO_{3/2}, hereinafter “T^{Pro}” units) and T^{Ph} units) (T^{Pro} units=30 mole %; T^{Ph} units=70 mole %)
(Mₙ: 1500; phenyl group content: 60 mass %; hydroxyl group content: 6 mass %; flake-like at room temperature);
silicone resin (CA-1): trimethoxysilicate resin that consists of trimethoxysiloxy units (consists of CH₃O)₃SiO_{1/2}, and SiO_{4/2} units) (flake-like at room temperature);
silicone resin (CA-2): propylsilsesquioxane that consists practically entirely of T^{Pro} units;
silicone resin (CA-3): phenylsilsesquioxane that consists practically entirely of T^{Ph} units (Mₙ: 5000; (flake-like at room temperature).

[Silicone Fluid]

The following silicone fluids were used for the preparation of the organopolysiloxane compositions shown in Table 1 (Samples 1 to 9):
(B-1): trimethylpentaphenyltrisiloxane (refractive index: 1.58; viscosity: 175 mPa·s; entirely transparent appearance);
(CB-1): phenyl(trimethylsiloxy)siloxane (refractive index: 1.56; viscosity: 20 mPa·s; entirely transparent appearance)

### Practical Examples 1 to 4; Comparative Examples 1 to 5

Viscosities, indices of refraction, and results of evaluations of appearances of the prepared organopolysiloxane compositions (Examples No. 1 to 9) are shown in Table 1. The silicone resin components and silicone oils components used in the preparation of compositions of Samples 1 to 9 were mechanically stirred and mixed at 120° C. until a uniform state was reached. If the uniform state was not reached after 30 min. of continuous stirring and mixing, the stirring was discontinued, and a sample was taken at this point.

### Practical Examples 1*, 2*

At the same structures as in Samples 1 and 2 shown in Table 1, silicone resin components were dissolved in equal quantities of toluene, and after uniform toluene solution were formed, silicone fluid components were added to the respective solutions and uniformly mixed with them. Following this, the toluene was removed by stripping under a reduced pressure, and, as a result, Samples 1* and 2* were obtained. In the method described above, there was no need in heating the samples to 120° C., and the obtained Samples 1* and 2* has the same appearances, viscosities, and indices of refraction as Samples 1 and 2 obtained in above-described Application Examples 1 and 2.

TABLE 1
Organopolysiloxane Compositions 1 to 9 and Results of their Evaluation

| | | Silicone | Silicone | | | | Cosmetic | |
|---|---|---|---|---|---|---|---|---|
| Appl. | | resin | fluid | Appear- | Refractive | | Formulation | |
| Example | No. | (mass %) | (mass %) | ance | index | Viscosity | No. 1 | No. 3 |
| Appl. | 1 | (A-1) | (B-1) | ◯ | 1.58 | 860 | ◯ | ◯ |
| Ex. 1 | | 20 | 80 | | | | | |
| Appl. | 2 | (A-1) | (B-1) | ◯ | 1.58 | 25,600 | — | — |
| Ex. 2 | | 40 | 60 | | | | | |
| Appl. | 3 | (A-1) | (B-1) | ◯ | 1.57 | >30000 | — | — |
| Ex. 3 | | 60 | 40 | | | | | |
| Appl. | 4 | (A-2) | (B-1) | ◯ | 1.57 | 8,200 | — | — |
| Ex. 4 | | 40 | 80 | | | | | |
| Comp. | 5 | (CA-1) | (B01) | X | — | — | — | — |
| Ex. 1 | | 20 | 80 | | | | | |
| Comp. | 6 | (CA-2) | (B01) | X | — | — | — | — |
| Ex. 2 | | 20 | 60 | | | | | |
| Comp. | 7 | (A-1) | (CB-1) | X | — | — | — | — |
| Ex. 3 | | 20 | 80 | | | | | |
| Comp. | 8 | (A-2) | (CB-1) | X | — | — | — | — |
| Ex. 4 | | 20 | 80 | | | | | |
| Comp. | 9 | (CA-3) | (B-1) | Δ | 1.58 | 860 | X | X |
| Ex. 5 | | 20 | 80 | | | | | |

As can be seen from Table 1, the compositions prepared from the phenylsilsequioxane with molecular weight of 1500 and the trimethylpentaphenyltrisiloxane (Practical Examples 1 to 4) comprise uniform phenyl-containing organopolysiloxane compositions that have a high index of refraction, facilitate control of viscosity of raw cosmetic materials, and when used as cosmetic materials, impart to them excellent properties.

On the other hand, in the case of comparative examples, except for the composition of Comparative Example 5, where the composition was prepared from the trimethylpentaphenyltrisiloxane and the phenylsilsequioxane with molecular weight of 5000, it was impossible to obtain a uniform mixture, and the obtained compositions were unsuitable for use as cosmetic raw materials. Furthermore, when the phenyl-containing organopolysiloxane composition of Comparative Example 5 was used for the preparation of a blush or a transparent lipstick (Prescription 1), the product could not provide a long-lasting sense of transparency and had properties inferior to the phenyl-containing organopolysiloxane composition of Practical Example 1.

<Methods for Evaluating Luster and Gloss of Compositions>

The following methods were used for evaluating luster (gloss)-holding characteristics of the phenyl-containing organopolysiloxane composition of the invention.

(1) The composition of Sample 1 (left) and the composition and the trimethylpentaphenyltrisiloxane shown in Table 1 were applied, each in the amount of 0.1 g onto the left side and onto the right side of urethane-coated wooden plates, respectively, and then sample plates were produced by forming the applied substances into circular shapes having diameters of 5 cm.
(2) The aforementioned sample plates were held for 6 days and 30 days intact at room temperature (25° C.) under wind-free conditions, and then photographs were taken directly after the application and 6 days later. Gloss conditions were observed and compared.

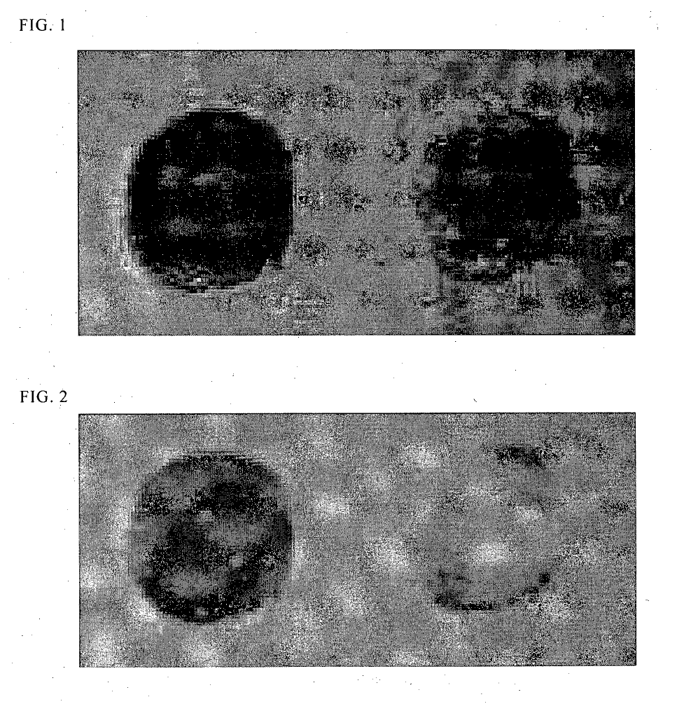

FIG. 1 shows the photograph of the sample plate taken directly after the application,

FIG. 2 shows the photograph taken in 6 days,

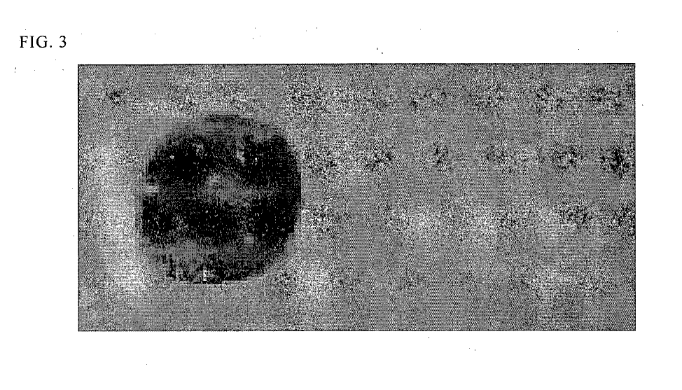

and FIG. 3 shows the photograph taken in 30 days.

It can be seen that, as compared to the phenyl-containing organopolysiloxane composition of Practical Example 1 where the cosmetic retained its gloss and luster characteristics (left side of the aforementioned sample plate), the composition of the Comparative Example almost completely lost its gloss characteristics and sense of moist touch was significantly reduced (right side of the aforementioned sample plate) (FIG. 2).

Furthermore, as compared to Sample 1 where the gloss, luster, and the sense of moist touch were preserved after the lapse of one month (left side of the sample plate), in the case of the comparative sample the gloss almost completely disappeared, and the sense of moist touch was significantly reduced (right side of the sample plate) (FIG. 3).

### Practical Example 5

A transparent lipstick was prepared from the components shown below (all quantities are in mass %):

### <Prescription 1: Transparent Lipstick>

| | | |
|---|---|---|
| (1) | polyamide-modified silicone*¹ | 16.5 |
| (2) | cyclohexane siloxane | 32.96 |
| (3) | isopropyl myristate | 22.42 |
| (4) | tri(caprylic acid/capric acid) glyceryl | 22.42 |
| (5) | phenyl-containing organopolysiloxane | 5.5 |
| | composition (No. 1) | |
| (6) | Pearle pigment*² | 0.2 |

*¹2-8178 Gellant, the product of Dow Corning Toray Co., Ltd.
*²Timiron ® Super Red, the product of Merck Co., Inc.

### (Method of Preparation)

Components (1) to (6) were dissolved at 100° C., uniformly mixed, poured into a lipstick mold, and after cooling turned into a transparent lipstick.

### (Evaluation Results)

The obtained transparent lipstick was entirely uniform, produced a sense of transparency, produced a luster effect in appearance, and therefore, as shown in Table 1, was evaluated with criterion [◯].

### Comparative Example 6

### Comparative Prescription

### Transparent Lipstick

A transparent lipstick was produced in the same manner as with aforementioned Prescription 1 except that the phenyl-containing organopolysiloxane composition No. 9 of Comparative Example 5 was used instead of phenyl-containing organopolysiloxane Composition No. 1 used in Prescription 1.

### (Evaluation Results)

The obtained transparent lipstick was not entirely uniform, did not produce a sense of transparency, and was inferior in luster to Prescription 1. Furthermore, after a lapse of one month, the components separated. Therefore, this lipstick was evaluated in Table 1 with criterion [X].

### Practical Example 6

A transparent lipstick was prepared from the components shown below (all quantities are in mass %):

### <Prescription 2: Eye Shadow>

| | | |
|---|---|---|
| (1) | polyester-modified silicone*¹ | 10 |
| (2) | cyclopentasiloxane | 13 |
| (3) | acryl-modified silicone*² | 30 |
| (4) | propyl-modified silicone*³ | 15 |
| (5) | phenyl-containing organopolysiloxane | 7 |
| | composition (No. 1) | |
| (6) | water | 1 |
| (7) | high-molecular-weight silicone emulsion*⁴ | 10 |
| (8) | pigment*⁵ | 2.2 |
| (9) | pearle pigment*⁶ | 1.3 |

*¹BY11-030, the product of Dow Corning Toray Co., Ltd.
*²FA 4001 CM, the product of Dow Corning Toray Co., Ltd.
*³670 Fluid, the product of Dow Corning Toray Co., Ltd.
*⁴HMW2220, the product of Dow Corning Toray Co., Ltd.
*⁵ColorOn ® 17323, the product of Merck Co., Inc.
*⁶Timiron ® Super Blue, the product of Merck Co., Inc.

### (Method of Preparation)

Components (1) to (5) were uniformly mixed at room temperature. While stirring the components at high speed, component (6) was slowly added, and the mixture was brought to a uniform state. The stirring speed was reduced, and component (7) was slowly added. The entire mixture was then uniformly mixed for another 10 min.

Following this, components (8) and (9) were added, and the product was mixed to uniformity.

### (Evaluation Results)

An eye shadow produced from the aforementioned prescription was entirely uniform and made it possible to obtain an eye shadow product that had excellent luster and gloss.

### Practical Example 7

A blush cosmetic was produced from components listed below. (all quantities are in mass %):

### <Prescription 3: Blush Cosmetic>

| | | |
|---|---|---|
| (1) | pigment*¹ | 20.0 |
| (2) | pearle pigment*² | 20.0 |
| (3) | phenyl-containing organopolysiloxane | 60 |
| | composition (No. 1) | |

*¹ColorOn ® Red Brown, the product of Merck Co., Inc.
*²Timiron ® Super Red, the product of Merck Co., Inc.

### (Method of Preparation)

Components (1) and (2) were uniformly mixed, Component (3) was added, and the content was mixed to uniformity.

### (Evaluation Results)

The blush cosmetic was obtained as a paste with uniform luster. Since this condition remained stable for more than one month, the product was evaluated in Table 1 with criterion [◯].

### Comparative Example 7

### Comparative Prescription

### Blush Cosmetic

A blush cosmetic was produced in the same manner as with aforementioned Prescription 3 except that the phenyl-containing organopolysiloxane composition No. 9 of Comparative Example 5 was used instead of phenyl-containing organopolysiloxane Composition No. 1 used in Prescription 1.

### (Evaluation Results)

By appearance, the obtained blush cosmetic was not different from one obtained with Prescription 3, but the oil phase gradually separated and, in a month, non uniformity occurred with the separation of the oil and paste layers. Therefore, in Table 1 the product is evaluated with criterion [X].

### Practical Example 8

A water-proof mascara was prepared from the following components (all quantities are in mass %):

### <Prescription 4: Water-Proof Mascara>

| | | |
|---|---|---|
| (1) | beeswax | 10 |
| (2) | candelilla wax | 7 |
| (3) | carnauba wax | 3 |
| (4) | stearic acid | 5 |
| (5) | glyceryl stearate | 5 |
| (6) | antiseptic | appropriate amount |
| (7) | water | balance |
| (8) | propylene glycol | 5 |
| (9) | triethanolamine | 1.5 |
| (10) | black iron oxide | 10 |
| (11) | acryl-modified silicone* | 5 |
| (12) | isododecane | 5 |
| (13) | phenyl-containing organopolysiloxane | 8 |
| | composition (No. 1)* | |

*FA 4002 ID, the product of Dow Corning Toray Co., Ltd.

### (Method of Preparation)

1. Components (1) to (6) were dissolved at 85° C.
2. Components (7) to (9) were dissolved at 80° C.
3. While stirring aforementioned 2 at high speed, aforementioned 1 is added and the content is emulsified.
4. While stirring aforementioned 3, component (10) is added.
5. The content was cooled to 50° C.
6. Components (11) to (13) was added to aforementioned 5, the content is mixed and then cooled to room temperature.

### (Evaluation Results)

The water-proof mascara produced from the aforementioned prescription was entirely uniform and comprised a glossy black paste composition.

### Practical Example 9

An emulsion-type mascara was prepared from the following components (all quantities are in mass %):

### <Prescription 5: Mascara (Emulsion Type)>

| | | |
|---|---|---|
| (1) | polyester-modified silicone*¹ | 10 |
| (2) | carbon black | 25 |
| (3) | silicone elastomer*² | 10 |
| (4) | cyclopentasiloxane (D5) | 10 |
| (5) | water | balance |
| (6) | sodium chloride | 2 |
| (7) | antiseptic | appropriate amount |
| (8) | acryl-modified silicone*² | 6.7 |
| (9) | cyclopentasiloxane (D5) | 6.7 |
| (10) | phenyl-containing organopolysiloxane | 6.6 |
| | composition (No. 1) | |

*¹BY22-008M, the product of Dow Corning Toray Co., Ltd.
*²9040 Silicone Elastomer Blend, the product of Dow Corning Toray Co., Ltd.
*³FA 4001 CM, the product of Dow Corning Toray Co., Ltd.

### (Method of Preparation)

1. Components (1) and (2) were mixed at 60° C.
2. Components (3) and (4) were mixed.
3. While stirring aforementioned 1 at high speed, aforementioned 2 was added.
4. The content was further mixed with components (5), (6), and (7).
5. While stirring aforementioned 3 at high speed, aforementioned 4 was added, and the content was emulsified.
6. The product was then cooled to 40° C.
7. Components (8), (9), (10) were added, and the content was mixed.
8. The content was cooled to room temperature.

### (Evaluation Results)

The emulsion-type mascara produced from the aforementioned prescription was entirely uniform.

1. A phenyl-containing organopolysiloxane composition that has an index of refraction of the entire composition that is equal to or greater than 1.50 and that has a viscosity of the entire composition at 25° C. in the range of 200 to 500,000 mPa·s.

2. The phenyl-containing organopolysiloxane composition according to claim 1, comprising:
100 parts by mass of a phenyl silsesquioxane resin (A) that has a number-average molecular weight measured by gel permeation chromatography (GPC) in the range of 500 to 2,000, wherein 15 or more mole % of siloxy-units in the resin structure is phenylsiloxy units represented by the following formula: (C₆H₅SiO_{3/2}); and
50 to 1,000 parts by mass of a phenyl-containing organopolysiloxane (B) (except for a organopolysiloxane corresponding to component (A)) that is liquid at 25° C., has an index of refraction equal to or greater than 1.45, and contains in its molecule phenylsiloxy units represented by structural formula (1): 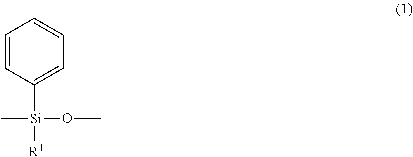
(where, R¹, each independently, is a substituted or unsubstituted univalent hydrocarbon group).

3. The phenyl-containing organopolysiloxane composition according to claim 2, wherein component (A) comprises a phenyl silsesquioxane resin that has a number-average molecular weight in the range of 750 to 1,800, wherein 40 or more mole % of siloxy-units in the resin structure is phenylsilsesquioxane units represented by the following formula: C₆H₅SiO_{3/2}; and wherein component (B) comprises a phenyl-containing organopolysiloxane represented by the following structural formula (1-1): 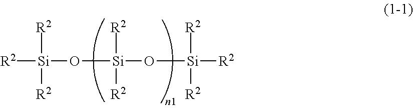
(wherein, R² are selected from phenyl groups, aralkyl groups, hydrogen atoms, hydroxyl groups, or alkyl or fluoro-alkyl groups having 1 to 20 carbon atoms; among these groups, the content of phenyl groups is at least 50% of total groups; and “n1” is a number in the range of 0 to 1000).

4. The phenyl-containing organopolysiloxane composition according to claim 1, wherein the index of refraction of the entire composition is in the range of 1.50 to 1.60, and wherein the viscosity of the entire composition at 25° C. in the range of 200 to 100,000 mPa·s.

5. The phenyl-containing organopolysiloxane composition according to claim 2, wherein component (A) comprises a phenyl silsesquioxane resin that has a number-average molecular weight in the range of 750 to 1,800 and that has one or more hydroxyl groups at molecular terminals; and wherein 60 to 100 mole % of siloxy-units in the resin structure is phenylsilsesquioxane units represented by the following formula: C₆H₅SiO_{3/2}.

6. The phenyl-containing organopolysiloxane composition according to claim 1, wherein component (B) comprises a phenyldisiloxane, phenyltrisiloxane, or a phenyltetrasiloxane represented by the following structural formula (1-2): 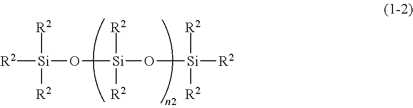
(where R² are the same groups as defined above; among these groups, the content of phenyl group is at least 50%; and “n” is a number in the range of 0 to 2).

7. The phenyl-containing organopolysiloxane composition according to claim 1, wherein component (B) is a trimethylpentaphenyl trisiloxane.

8. A cosmetic raw material comprising the phenyl-containing organopolysiloxane composition according to claim 1.

9. A cosmetic material comprising the phenyl-containing organopolysiloxane composition according to claim 1.

10. A makeup-type cosmetic material comprising the phenyl-containing organopolysiloxane composition according to claim 1 and at least one type of a powder or a colorant.

11. A method of producing a phenyl-containing organopolysiloxane composition according to claim 1, the method comprising the steps of:
(I) mixing components (A) and (B) in the presence of an organic solvent; and
(II) removing the organic solvent from the mixture upon completion of Step (I).

12. A method of adjusting viscosity of phenyl-containing organopolysiloxane according to claim 1, wherein the viscosity of the composition at 25° C. is adjusted in the range of 200 to 100,000 mPa·s by adjusting the mixture ratio of component (A) to component (B) in the range of (1:0.5) to (1:10).

13. The phenyl-containing organopolysiloxane composition according to claim 2, wherein the index of refraction of the entire composition is in the range of 1.50 to 1.60, and wherein the viscosity of the entire composition at 25° C. in the range of 200 to 100,000 mPa·s.

14. The phenyl-containing organopolysiloxane composition according to claim 3, wherein component (A) comprises a phenyl silsesquioxane resin that has a number-average molecular weight in the range of 750 to 1,800 and that has one or more hydroxyl groups at molecular terminals; and wherein 60 to 100 mole % of siloxy-units in the resin structure is phenylsilsesquioxane units represented by the following formula: C₆H₅SiO_{3/2}.

15. The phenyl-containing organopolysiloxane composition according to claim 2, wherein component (B) comprises a phenyldisiloxane, phenyltrisiloxane, or a phenyltetrasiloxane represented by the following structural formula (1-2): 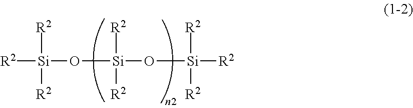
(where R² are the same groups as defined above; among these groups, the content of phenyl group is at least 50%; and “n” is a number in the range of 0 to 2).
